(19) United States
(12) Patent Application Publication
(10) Pub. No.: US 2021/0163410 A1
(43) Pub. Date: Jun. 3, 2021

(54) AN IMPROVED PROCESS FOR THE PREPARATION OF (2S)-2-[(4R)-2-OXO-4-PROPYLTETRAHYDRO-1H-PYRROL-1-YL] BUTANAMIDE AND ITS INTERMEDIATES THEREOF

(71) Applicants: Thirumalal Rajan SRINIVASAN, Hyderabad (IN); MSN LABORATORIES PRIVATE LIMITED, R&D CENTER, Sangareddy (Dist), Hyderabad, Telangana (IN)

(72) Inventors: Thirumalai Rajan SRINIVASAN, Hyderabad (IN); Eswaraiah SAJJA, Hyderabad (IN); Satyanarayana REVU, Hyderabad (IN); Prabhakar CHERKUPALLY, Hyderaba (IN)

(73) Assignee: MSN LABORATORIES PRIVATE LIMITED, R&D CENTER, Sangareddy (Dist), Hyderabad, Telangana (IN)

(21) Appl. No.: 16/618,328

(22) PCT Filed: May 29, 2018
(86) PCT No.: PCT/IN2018/050344

§ 371 Date: Nov. 29, 2019

(30) Foreign Application Priority Data
May 29, 2017 (IN) 201741018764
Jan. 20, 2018 (IN) 201841002418

(51) Int. Cl. C07D 207/27

(52) U.S. Cl. CPC C07D 207/27

(57) ABSTRACT

The present invention relates to an improved process for the preparation of (2S)-2-[(4R)-2-oxo-4-propyltetrahydro-1H-pyrrol-1-yl] butanamide compound of formula-1, its intermediates, novel salt compounds of intermediates of the compound of formula-1. Further the use of novel salts in the preparation of the compound of formula-1. The present invention also relates to the novel process for the preparation of the compound of formula-1.

### RELATED APPLICATION

This application is a U.S. National Phase Entry of the International Patent Application Number PCT/IN2018/050344, filed on May 29, 2018, which claims the benefit of priority of Indian Patent Application Number 201741018764 filed on May 29, 2017 and Indian Patent Application Number 201841002418 filed on Jan. 20, 2018, both of which are incorporated herein by reference, in their entirety.

### FIELD OF THE INVENTION

The present invention relates to an improved process for the preparation of (2S)-2-[(4R)-2-oxo-4-propyltetrahydro-1H-pyrrol-1-yl] butanamide which is represented by the following structural formula-1.

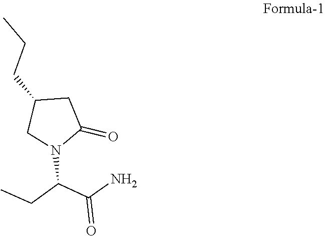

Further present invention relates to novel processes for the preparation of (2S)-2-[(4R)-2-oxo-4-propyltetrahydro-1H-pyrrol-1-yl] butanamide which is represented by the following structural formula-1.

The present invention also relates to the novel intermediate compounds which are useful in the preparation of the compound of formula-1.

### BACKGROUND OF THE INVENTION

Brivaracetam is chemically known as (2S)-2-[(4R)-2-oxo-4-propyltetrahydro-1H-pyrrol-1-yl] butanamide which was approved in US and Europe under the brand name of Briviact. It is indicated as adjunctive therapy in the treatment of partial-onset seizures in patients 16 years of age and older with epilepsy.

(2S)-2-[(4R)-2-oxo-4-propyltetrahydro-1H-pyrrol-1-yl] butanamide compound of formula-1 was first disclosed in U.S. Pat. No. 6,911,461 (herein after referred as US'461). US'461 also generically discloses the preparation of (2S)-2-[(4R)-2-oxo-4-propyltetrahydro-1H-pyrrol-1-yl] butanamide compound of formula-1 and its intermediate. US'461 discloses the purification of the compound of formula-1 is using by column chromatography.

US762947B2, U.S. Pat. No. 8,076,493B2 & U.S. Pat. No. 8,338,621B2 discloses the process for the preparation of the compound of formula-1 via novel intermediates through novel process.

All the above processes disclose the preparation of the pure compound of formula-1 involving the separation of enantiomers of the compound of formula-1 using column chromatography, using expensive reagents, cumbersome workups and further involving column chromatography for purification of final drug substance which is tedious and time consuming and making them uneconomical and provides compound formula-1 with low yield and less purity.

However, in view of the above drawbacks there is still an unmet need in the art to develop an improved, economical, eco-friendly process for the preparation of compound of formula-1 with high yield and purity.

In the present invention, stereospecific starting materials have been used for the preparation of compound of formula-1. The use of stereospecific starting materials has an advantage that they do not provide racemic products in the subsequent reactions and also do not form impurities. Therefore, there is a very little need of purifications ate the various stages which not only avoids the use of methods like column chromatography which are very cumbersome and not practicable at the commercial levels.

The present inventors developed a novel process for the preparation of compound of formula-1 using novel key starting materials. The compound of formula-1 prepared according to the present invention provides highly pure compound with a good yield.

### BRIEF DESCRIPTION OF THE INVENTION

The first aspect of the present invention is to provide an improved process for the preparation the compound of formula-1.

The second aspect of the present invention is to provide an improved process for the preparation of the compound of formula-3.

The third aspect of the present invention is to provide purification of the (R)-2-(2-(tert-butoxy)-2-oxoethyl)pentanoic acid compound of formula-5.

The fourth aspect of the present invention is to provide an improved process for the preparation of the compound of formula-5.

The fifth aspect of the present invention is to provide crystalline compound of general formula-9 and process for the preparation thereof.

The sixth aspect of the present invention is to provide an improved process for the preparation of the compound of formula-2.

The seventh aspect of the present invention is to provide an improved process for the preparation of the compound of formula-1.

The eight aspect of the present invention is to provide a novel process for the preparation of (2S)-2-[(4R)-2-oxo-4-propyltetrahydro-1H-pyrrol-1-yl] butanamide compound of formula-1.

The ninth aspect of the present invention is to provide an alternative process for the preparation of (2S)-2-[(4R)-2-oxo-4-propyltetrahydro-1H-pyrrol-1-yl] butanamide compound of formula-1.

The tenth aspect of the present invention is to provide an alternative process for the preparation of (2S)-2-[(4R)-2-oxo-4-propyltetrahydro-1H-pyrrol-1-yl] butanamide compound of formula-1.

The eleventh aspect of the present invention is to provide a novel process for the preparation of (2S)-2-[(4R)-2-oxo-4-propyltetrahydro-1H-pyrrol-1-yl] butanamide compound of formula-1.

The twelfth aspect of the present invention is to provide the novel process for the preparation of (2S)-2-[(4R)-2-oxo-4-propyltetrahydro-1H-pyrrol-1-yl] butanamide compound of formula-1.

### BRIEF DESCRIPTION OF THE DRAWINGS

FIG. 1: Illustrates the PXRD pattern of (2S)-2-[(4R)-2-oxo-4-propyltetrahydro-1H-pyrrol-1-yl] butanamide compound of formula-1.

FIG. 2: Illustrates the DSC histogram of (2S)-2-[(4R)-2-oxo-4-propyltetrahydro-1H-pyrrol-1-yl] butanamide compound of formula-1.

FIG. 3: Illustrates the PXRD pattern of benzylamine salt of (R)-2-(2-(tert-butoxy)-2-oxoethyl)pentanoic acid compound of formula-9a.

FIG. 4: Illustrates the PXRD pattern of tertiarybutyl amine salt of (R)-2-(2-(tert-butoxy)-2-oxoethyl)pentanoic acid compound of formula-9b.

FIG. 5: Illustrates the PXRD pattern of (S)-phenylethyl amine salt of (R)-2-(2-(tert-butoxy)-2-oxoethyl)pentanoic acid compound of formula-9c.

FIG. 6: Illustrates the PXRD pattern of tertiarybutyl amine salt of (R)-2-(2-(tert-butoxy)-2-oxoethyl)pentanoic acid compound of formula-9b obtained according to example-17.

FIG. 7: Illustrates the DSC histogram of tertiarybutyl amine salt of (R)-2-(2-(tert-butoxy)-2-oxoethyl)pentanoic acid compound of formula-9b obtained according to example-17.

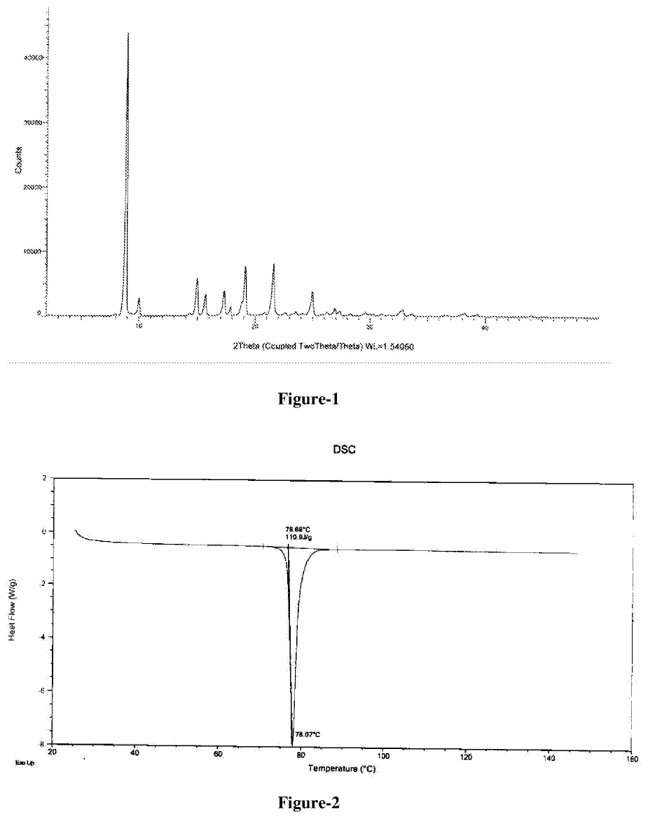

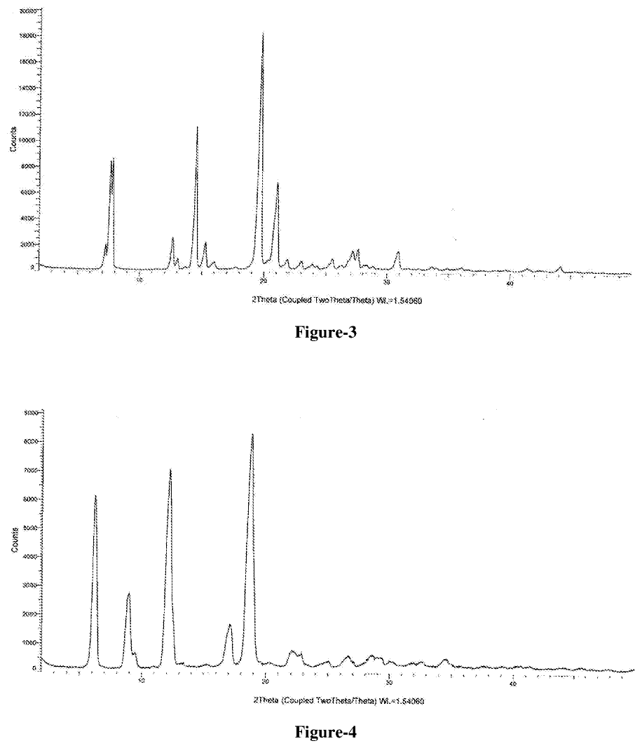

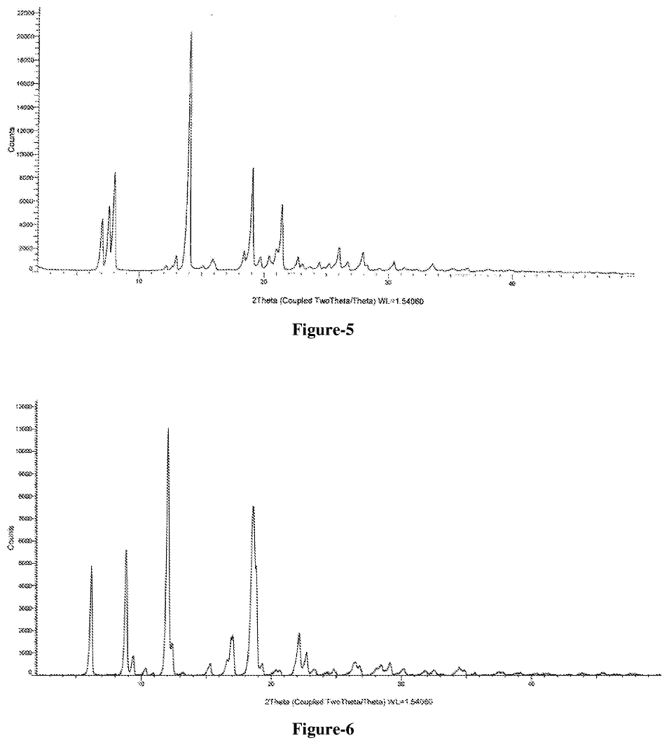

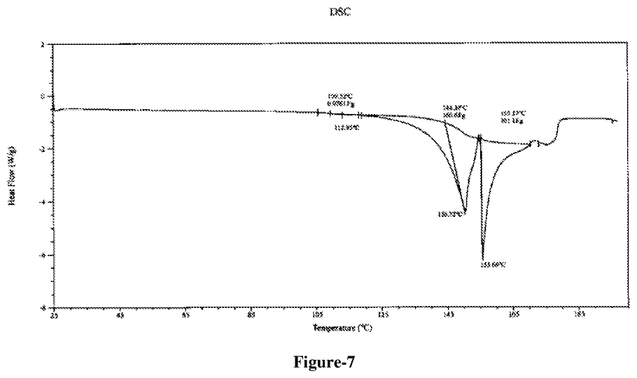

### DETAILED DESCRIPTION OF THE INVENTION

As used herein the term “suitable solvent” used in the present invention refers to “hydrocarbon solvents” such as n-hexane, n-heptane, cyclohexane, pet ether, benzene, toluene, pentane, cycloheptane, methyl cyclohexane, ethylbenzene, m-, o-, or p-xylene, or naphthalene and the like; “ether solvents” such as dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, 1,2-dimethoxy ethane and the like; “ester solvents” such as methyl acetate, ethyl acetate, isopropyl acetate, n-butyl acetate and the like; “polar-aprotic solvents such as dimethylacetamide (DMA), dimethylformamide (DMF), dimethylsulfoxide (DMSO), N-methylpyrrolidone (NMP) and the like; “chloro solvents” such as dichloromethane, dichloroethane, chloroform, carbon tetrachloride and the like; “ketone solvents” such as acetone, methyl ethyl ketone, methyl isobutylketone and the like; “nitrile solvents” such as acetonitrile, propionitrile, isobutyronitrile and the like; “alcohol solvents” such as methanol, ethanol, n-propanol, isopropanol, n-butanol, isobutanol, t-butanol, 2-nitroethanol, 2-fluoroethanol, 2,2,2-trifluoroethanol, ethylene glycol, 1,2-propanediol (propylene glycol), 2-methoxyethanol, 1, 2-ethoxyethanol, diethylene glycol, 1, 2, or 3-pentanol, neo-pentyl alcohol, t-pentyl alcohol, diethylene glycol monoethyl ether, cyclohexanol, benzyl alcohol, phenol, or glycerol and the like; “polar solvents” such as water or mixtures thereof.

As used herein the present invention the term “suitable base” refers to inorganic bases like “alkali metal carbonates” such as sodium carbonate, potassium carbonate, lithium carbonate and the like; “alkali metal bicarbonates” such as sodium bicarbonate, potassium bicarbonate and the like; “alkali metal hydroxides” such as sodium hydroxide, potassium hydroxide, lithium hydroxide and the like; alkali metal hydrides such as sodium hydride, potassium hydride, lithium hydride and the like; alkali metal amides such as sodium amide, potassium amide, lithium amide and the like; and organic bases like “alkali metal alkoxides” such as sodium methoxide, sodium ethoxide, potassium methoxide, potassium ethoxide, sodium tert.butoxide, potassium tert.butoxide, lithium tert.butoxide and the like; dimethylamine, diethylamine, diisopropyl amine, diisopropylethylamine, diisobutylamine, triethylamine, tertiary butyl amine, benzyl amine, pyridine, 4-dimethylaminopyridine (DMAP), N-methyl morpholine (NMM), 2,6-lutidine, lithium diisopropylamide; organosilicon bases such as lithium hexamethyldisilazide (LiHMDS), sodium hexamethyldisilazide (NaHMDS), potassium hexamethyldisilazide (KHMDS) or mixtures thereof.

As used herein the present invention the term “acid” refers to inorganic acids selected from hydrochloric acid, hydrobromic acid, sulfuric acid, nitric acid, phosphoric acid etc; organic acids such as acetic acid, maleic acid, malic acid, tartaric acid, oxalic acid, trifluoroacetic acid, methane sulfonic acid, p-toluene sulfonic acid; chiral acids such as S-(+) mandelic acid, R-(−) mandelic acid, L-(+)tartaric acid, D-(−)tartaric acid, L-malic acid, D-malic acid, D-maleic acid, (−)-naproxen, (+)-naproxen, (1R)-(−)-camphor sulfonic acid, (1S)-(+)-camphor sulfonic acid, (1R)-(+)-bromocamphor-10-sulfonic acid, (1S)-(−)-bromocamphor-10-sulfonic acid, (−)-Dibenzoyl-L-tartaric acid, (−)-Dibenzoyl-L-tartaricacid monohydrate, (+)-Dibenzoyl-D-tartaric acid, (+)-Dibenzoyl-D-tartaric acid monohydrate, (+)-dipara-tolyl-D-tataric acid, (−)-dipara-tolyl-L-tataricacid, L(−)-pyroglutamic acid, L(+)-pyroglutamic acid, (−)-lactic acid, L-lysine, D-lysine etc., and like.

The term “salts” used in the present invention refers to acid addition salts selected from inorganic acids such as hydrochloric acid, hydrobromic acid, sulfuric acid, nitric acid, phosphoric acid; organic acids such as acetic acid, maleic acid, malic acid, oxalic acid, trifluoroacetic acid, methane sulfonic acid, p-toluene sulfonic acid; chiral acids such as S-(+) mandelic acid, R-(−) mandelic acid, L-(+)tartaric acid, D-(−)tartaric acid, L-malic acid, D-malic acid, D-maleic acid, (−)-naproxen, (+)-naproxen, (1R)-(−)-camphor sulfonic acid, (1S)-(+)-camphor sulfonic acid (1R)-(+)-bromocamphor-10-sulfonic acid, (1S)-(−)-bromocamphor-10-sulfonic acid, (−)-Dibenzoyl-L-tartaric acid, (−)-Dibenzoyl-L-tartaricacid monohydrate, (+)-Dibenzoyl-D-tartaric acid, (+)-Dibenzoyl-D-tartaric acid monohydrate, (+)-dipara-tolyl-D-tataric acid, (−)-dipara-tolyl-L-tataricacid, L(−)-pyroglutamic acid, L(+)-pyroglutamic acid, (−)-lactic acid, L-lysine, D-lysine etc., and like.

The term “condensing agent or coupling agent” used in the present invention is selected form N,N-dicyclohexylcarbodiimide (DCC), N,N′-diisopropylcarbodiimide (DIC), carbonyldiimidazole (CDI), 1-ethyl-3-(3-dimethylaminopropyl)carbodiimide hydrochloride (EDC.HCl), 0-(7-aza-benzotriazole-1-yl)-N,N,N′,N′-tetramethyl uronium hexafluoro phosphate (HATU), alkyl or aryl chloroformates such as ethyl chloroformate, benzylchloroformate, diphenylphosphoroazidate (DPPA), thionyl chloride, pivalyl chloride, oxalyl chloride, phosphorous oxychloride, phosphorous pentachloride, 4-methyl-2-oxopentanoyl chloride (i-BuCOCOCl), benzotriazol-1-yl-oxytripyrrolidino phosphonium hexafluorophosphate (PyBOP), methane sulfonyl chloride and the like; optionally in combination with 1-hydroxy-7-azatriazole (HOAt), 1-hydroxybenzotriazole (HOBt), 1-hydroxy-1H-1,2,3-triazole-4-carboxylate (HOCt), O-(benzotriazol-1-yl)-N,N,N,N-tetramethyluronium tetrafluoroborate (TBTU), N-hydroxysuccinamide (HOSu), N-hydroxysulfosuccinimide (Sulfo-NHS), 4-dimethylaminopyridine (DMAP).

The term “reducing agent” used in the present invention includes, for example LAH, NaBH₄, sodium cyanoborohydride, KBH₄, mixture of NaBH₄ & acetic acid, mixture of NaBH₄ & trifluoroacetic acid, mixture of NaBH₄& iodine, mixture of NaBH₄ & trimethylchlorosilane, mixture of NaBH₄ & magnesium chloride, mixture of NaBH₄ & calcium chloride, mixture of NaBH₄ & one of transition metal chlorides, boranes, Vitride {=Sodium bis(2-methoxyethoxy)aluminumhydride} and like; and hydrogen in presence of a catalyst which include, without limitation heterogeneous catalysts containing from about 0.1% to about 20% by weight of transition metals such as Ni, Pd, Pt, Rh, Re, Ru and Ir, including oxides and combination thereof, which are typically supported on various materials including Al₂O₃, C, CaCO₃, SrCO₃, BaSO₄, MgO, SiO₂, TiO₂, ZrO₂ and the like. Many of these metals including Pd may be doped with an amine, sulfide or a second metal such as Pb, Cu and Zn. Useful catalysts include Raney nickel, palladium catalyst such as Pd/C, Pd/SrC₃, Pd/Al₂O₃, Pd/MgO, Pd/CaCO₃, Pd/BaSO₄, PdO, Pd Black, PdCl₂ and the like. Other useful catalysts Rh/C, Ru/C, Re/C, PtO₂, Rh/C, RuO₂. The reaction is typically carried out in the presence of one or more solvents including without limitation water, alcohols, ethers, ester, ketones, acids and hydrocarbon solvents such as, methanol, ethanol, isopropyl alcohol, tetrahydrofuran, ethyl acetate, acetic acid, dichloromethane and the like.

The first aspect of the present invention provides an improved process for the preparation of the compound of formula-1, comprising of,
a) reacting the compound of general formula-2 with a suitable halogenating agent in presence or absence of a catalyst in a suitable solvent to provide the compound of general formula-3, (or) reacting the compound of general formula-2 with a suitable sulphonyl chloride in presence or absence of base in a suitable solvent to provide the compound of general formula-3,
b) reacting the compound of general formula-3 with (S)-2-aminobutyramide or its salts in presence of a suitable base and optionally in presence of a catalyst in a suitable solvent to provide the compound of general formula-4,
c) treating the compound of general formula-4 with a suitable acid in presence or absence of a coupling agent to provide the compound of formula-1,
d) optionally purifying the compound of formula-1 using a suitable solvent.
   Wherein in step-a) the suitable halogenating agent is selected from TMSI, TMSCI, TMSBr, HBr, HCl or HI; the catalyst is sodium iodide, potassium iodide, calcium iodide, sodium bromide, potassium bromide, lithium chloride, lithium bromide; the suitable sulphonyl chloride is methane sulphonyl chloride, p-toluene sulphonyl chloride; the suitable solvent is selected from alcohol solvent; the suitable base is selected from inorganic bases or organic bases;
   in step-b) the suitable base is selected from inorganic bases or organic bases; the catalyst is sodium iodide, potassium iodide, calcium iodide, sodium bromide, potassium bromide or a phase transfer catalyst; the suitable solvent is selected from alcohol solvents, chloro solvents, ester solvents, ketone solvents, ether solvents, hydrocarbon solvents, nitrile solvents, polar aprotic solvents, polar solvents such as water and mixtures thereof;
   in step-c) the suitable acid is organic acids or inorganic acids, preferably organic acids; most preferably acetic acid; the coupling agent is as defined above; the suitable solvent is selected from alcohol solvents, chloro solvents, ester solvents, ketone solvents, ether solvents, hydrocarbon solvents, nitrile solvents, polar aprotic solvents, polar solvents such as water and mixtures thereof.

Preferred embodiment of the present invention provides an improved process for the preparation of the compound of formula-1, comprising of,
a) reacting the compound of formula-2a with a trimethylsillyl chloride [TMSCI] in presence of sodium bromide in ethanol to provide the compound of formula-3a,
b) reacting the compound of formula-3a with (S)-2-aminobutyramide hydrochloride in presence of potassium carbonate and potassium iodide in the mixture of tetrahydrofuran and dimethyl sulfoxide to provide the compound of formula-4a,
c) treating the compound of formula-4a with acetic acid in tetrahydrofuran to provide the compound of formula-1,
d) purifying the compound of formula-1 obtained in step-c) using diisopropyl ether or methyl tertiary butyl ether to get pure compound of formula-1.

Another preferred embodiment of the present invention provides an improved process for the preparation of compound of formula-1, comprising of,
a) reacting the compound of formula-2a with a trimethylsillyl bromide in ethanol to provide the compound of formula-3a,
b) reacting the compound of formula-3a with (S)-2-aminobutyramide hydrochloride in presence of potassium carbonate and potassium iodide in the mixture of tetrahydrofuran and dimethyl sulfoxide to provide the compound of formula-4a,
c) treating the compound of formula-4a in-situ with acetic acid in ethyl acetate to provide the compound of formula-1,
d) purifying the compound of formula-1 obtained in step-c) using diisopropyl ether to get pure compound of formula-1.

The above first aspect is schematically represented as follows:

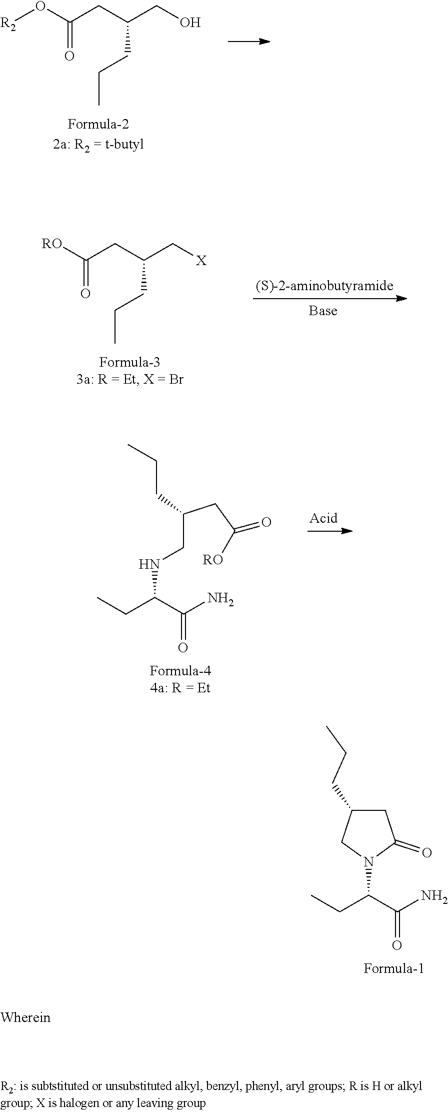

The second aspect of the present invention provides an improved process for the preparation of the compound of general formula-3, comprising of, reacting the compound of general formula-2 with a suitable halogenating agent or with a suitable sulphonyl chloride in presence or absence of a catalyst and a base in a suitable solvent to provide the compound of general formula-3; wherein the suitable halogenating agent is TMSI, TMSCI, TMSBr, HBr, HCl or HI; the catalyst is sodium iodide, potassium iodide, calcium iodide, sodium bromide, potassium bromide; the suitable sulphonyl chloride is methane sulphonyl chloride, p-toluene sulphonyl chloride; the suitable base is inorganic bases or organic bases and the suitable solvent is selected from alcohol solvents.

Preferred embodiment of the present invention provides an improved process for the preparation of the compound of formula-3a, comprising of, reacting the compound of formula-2a with trimethylsillyl chloride [TMSCI] in presence of sodium bromide in ethanol to provide the compound of formula-3a.

Further preferred embodiment of the present invention provides an improved process for the preparation of the compound of formula-3a, comprising of, reacting the compound of formula-2a with trimethylsillyl bromide [TMSBr] in ethanol to provide the compound of formula-3a.

The third aspect of the present invention provides purification of the (R)-2-(2-(tert-butoxy)-2-oxoethyl)pentanoic acid compound of general formula-5, comprising of,
a) treating the compound of general formula-5 with a suitable base in a suitable solvent to provide the compound of general formula-9,
b) treating the compound of general formula-9 with a suitable acid in a suitable solvent to provide the pure compound of formula-5.
   Wherein in step-a) the suitable base is selected from organic bases such as dimethylamine, diethylamine, diisopropyl amine, diisopropylethylamine, diisobutylamine, triethylamine, tertiary butyl amine, benzyl amine, pyridine; chiral bases such as (S)-phenylethyl amine, (S)-2-amino-3-methylbutane, (S)-4-Chloro-α-methylbenzylamine, (S)-(−)-N,α-Dimethylbenzylamine, (S)-(−)-N,N-Dimethyl-1-phenylethylamine, (S)-(−)-1-(2-Naphthyl)ethylamine and but not limited to;
   in step-b) the suitable acid is selected from inorganic or organic acid;
   in step-a) and step-b) the suitable solvent is selected from alcohol solvents, chloro solvents, ester solvents, ketone solvents, ether solvents, hydrocarbon solvents, nitrile solvents, polar solvents, water and mixtures thereof.

Preferred embodiment of the present invention provides purification of the (R)-2-(2-(tert-butoxy)-2-oxoethyl)pentanoic acid compound of formula-5a, comprising of,
a) treating the compound of formula-5a with benzyl amine in ethyl acetate to provide the compound of formula-9a,
b) treating the compound of formula-9a with hydrochloric acid in the mixture of water and toluene to provide the pure compound of formula-5a.

Another preferred embodiment of the present invention provides purification of the (R)-2-(2-(tert-butoxy)-2-oxoethyl)pentanoic acid compound of formula-5a, comprising of,
a) treating the compound of formula-5a with tertiary butyl amine in ethyl acetate to provide the compound of formula-9b,
b) treating the compound of formula-9b with hydrochloric acid in the mixture of water and toluene to provide the pure compound of formula-5a.

The fourth aspect of the present invention provides an improved process for the preparation of the compound of general formula-5, comprising of:
a) reacting the compound of general formula-6 with valeric acid in presence of a suitable coupling agent and a suitable base or catalyst in a suitable solvent to provide the compound of general formula-7,
b) reacting the compound of general formula-7 with the compound of general formula-10 in presence of a suitable base in a suitable solvent to provide the compound of general formula-8,
c) treating the compound of general formula-8 with hydrogen peroxide in presence of a suitable base in a suitable solvent to provide the compound of general formula-5,
d) optionally purifying the compound of general formula-5.
   Wherein in step-a) the suitable coupling agent is same as defined above;
   in steps-a) to c) the suitable base is selected from inorganic or organic bases; the suitable solvent is selected from alcohol solvents, chloro solvents, ester solvents, ketone solvents, ether solvents, hydrocarbon solvents, nitrile solvents, polar solvents, water and mixtures thereof.

Preferred embodiment of the present invention provides an improved process for the preparation of the compound of formula-5a, comprising of,
a) reacting the compound of formula-6a with valeric acid in presence of dicyclohexylcarbodiimide and dimethylaminopyridine in dichloromethane to provide the compound of formula-7a,
b) reacting the compound of formula-7a with tertiary butyl bromoacetate compound of formula-10a in presence of NaHMDS in tetrahydrofuran to provide the compound of formula-8a,
c) treating the compound of formula-8a with hydrogen peroxide in presence of sodium hydroxide in the mixture of water and tetrahydrofuran to provide the compound of formula-5a,
d) purifying the compound of formula-5a by treating with benzyl amine followed by with hydrochloric acid to get pure compound of formula-5a.

Another preferred embodiment of the present invention provides an improved process for the preparation of the compound of formula-5a, comprising of,
a) reacting the compound of formula-6a with valeric acid in presence of dicyclohexylcarbodiimide and dimethylaminopyridine in dichloromethane to provide the compound of formula-7a,
b) reacting the compound of formula-7a with tertiary butyl bromoacetate compound of formula-10a in presence of NaHMDS in tetrahydrofuran to provide the compound of formula-8a,
c) treating the compound of formula-8a with hydrogen peroxide in presence of sodium hydroxide in the mixture of water and tetrahydrofuran to provide the compound of formula-5a,
d) purifying the compound of formula-5a by treating with tertiary butylamine followed by with hydrochloric acid to get pure compound of formula-5a.
   The third & fourth aspects are schematically represented as follows:

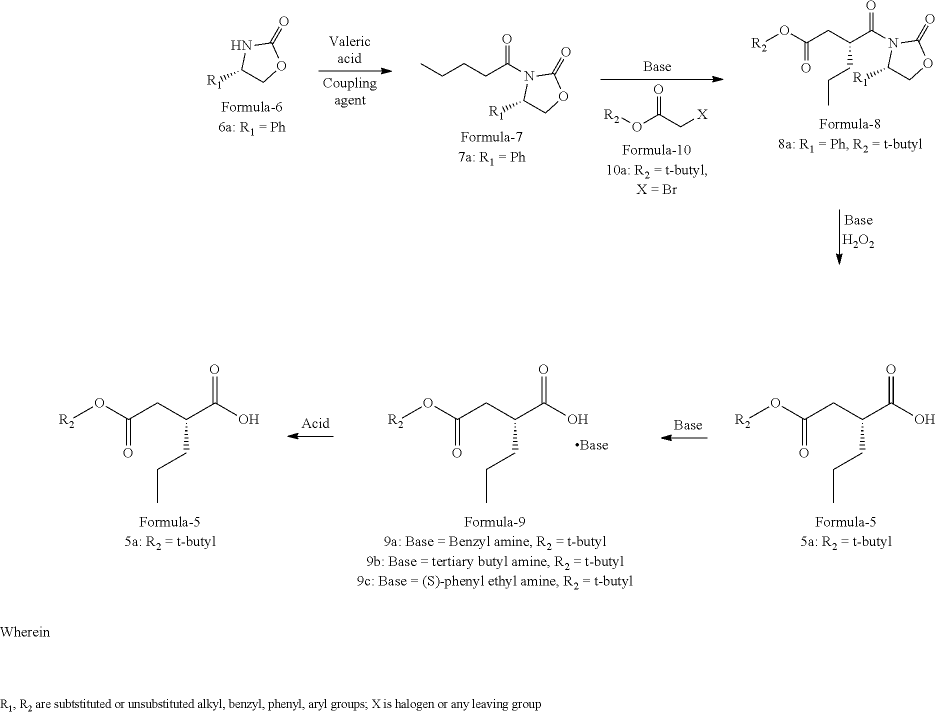

The compounds of general formula-5 and general formula-9 prepared according to the present invention are useful in the preparation of highly pure compound of formula-1 which is having chiral purity >99%, preferably 99.5%, more preferably 99.9% and purity >95%, preferably >98%, more preferably 99% and most preferably >99.5% by GC or HPLC.

The fifth aspect of the present invention provides crystalline compounds of general formula-9.

Further aspect of the present invention provides the novel base salt compounds of general formula-9.

Further aspect of the present invention provides process for the preparation of crystalline compound of general formula-9, comprising of, treating the compound of general formula-5 with a suitable base in a suitable solvent to provide the corresponding salt compound of general formula-9; wherein the suitable base and solvent is same as defined in step-a) of third aspect.

Preferred embodiment of the present invention provides a process for the preparation of crystalline compound of formula-9a, comprising of, treating the compound of formula-5a with benzyl amine in ethyl acetate to provide the compound of formula-9a which is further characterized by its powder X-Ray diffractogram having peaks at 7.7, 12.6, 14.5, 15.3, 19.7 and 21.01±0.2 degrees of two theta & is depicted in figure-3.

Another preferred embodiment of the present invention provides a process for the preparation of crystalline compound of formula-9b, comprising of, treating the compound of formula-5a with tertiary butyl amine in ethyl acetate to provide the compound of formula-9b which is further characterized by its powder X-Ray diffractogram having peaks at 6.2, 8.8, 12.1, 17.0, 18.6 and 22.1±0.2 degrees of two theta & is depicted in figure-4.

Another preferred embodiment of the present invention provides a process for the preparation of the compound of formula-9c, comprising of, treating the compound of formula-5a with (S)-phenyl ethyl amine in methyl tertiary butyl ether to provide the compound of formula-9c which is further characterized by its powder X-Ray diffractogram as depicted in figure-5.

The sixth aspect of the present invention provides an improved process for the preparation of the compound of general formula-2, comprising of, reduction of the compound of general formula-5 with a suitable reducing agent in a suitable solvent to provide the compound of general formula-2.

Preferred embodiment of the present invention provides an improved process for the preparation of the compound of formula-2a, comprising of, reduction of the compound of formula-5a with the mixture of sodium borohydride, BF₃-Et₂O in tetrahydrofuran to provide the compound of formula-2a.

Another preferred embodiment of the present invention provides an improved process for the preparation of the compound of formula-2a, comprising of, reduction of the compound of formula-5a with borane dimethyl sulfide complex in tetrahydrofuran to provide the compound of formula-2a.

The sixth aspect of the present invention is schematically represented as follows:

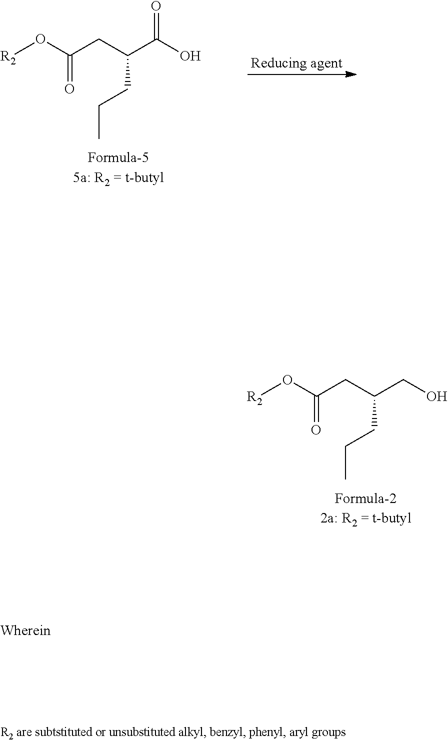

The seventh aspect of the present invention provides an improved process for the preparation of the compound of formula-1; comprising of:
a) treating the compound of general formula-2 with a suitable halogenating agent or with a suitable sulphonyl chloride in presence or absence of a catalyst and base in a suitable solvent to provide the compound of general formula-11,
b) reacting the compound of general formula-11 with (S)-2-aminobutyramide or its salts in presence of a suitable base and optionally in presence of a catalyst in a suitable solvent to provide the compound of general formula-12,
c) converting the compound of general formula-12 into the compound of formula-1,
d) optionally purifying the compound of formula-1 using a suitable solvent.
   Wherein in step-a) the suitable halogenating agent is selected from TMSI, TMSCI, TMSBr, HBr, HCl or HI; the catalyst is selected from sodium iodide, potassium iodide, calcium iodide, sodium bromide, potassium bromide, lithium chloride, lithium bromide; the suitable sulphonyl chloride is selected from methane sulphonyl chloride, p-toluene sulphonyl chloride; the suitable solvent is selected from alcohol solvents; the suitable base is selected from inorganic bases or organic bases;
   in step-b) the suitable base is selected from inorganic bases or organic bases; the suitable catalyst is selected from sodium iodide, potassium iodide, calcium iodide, sodium bromide, potassium bromide or a phase transfer catalyst; the suitable solvent is selected from alcohol solvents, chloro solvents, ester solvents, ketone solvents, ether solvents, hydrocarbon solvents, nitrile solvents, polar aprotic solvents, polar solvents such as water and mixtures thereof.

The seventh aspect of the present invention is schematically represented as follows:

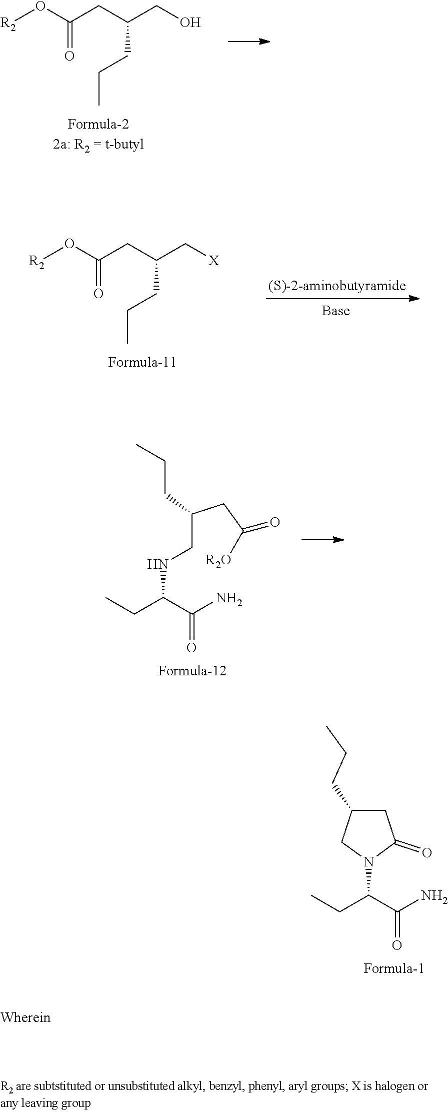

The eighth aspect of present invention provides a novel process for the preparation of compound of formula-1 comprising of the following steps,
a) reacting the compound of general formula-2 with a suitable oxidizing agent optionally in presence of base to provide the compound of general formula-13,
b) reacting the compound of general formula-13 with the (S)-2-amino butyramide compound of formula-18 or its salts in presence or absence of a base in a suitable solvent and a suitable reducing agent to provide the compound of general formula-12, or reacting the compound of general formula-13 with (S)-2-amino butyramide compound of formula-18 or its salts in the presence of a base in a suitable solvent followed by treating the obtained compound with a suitable reducing agent to provide the compound of general formula-12,
c) optionally purifying the compound of general formula-12 obtained in step-b),
d) cyclizing the compound of general formula-12 in the presence or absence of a catalyst in a suitable solvent to provide the compound of formula-1, or hydrolyzing the compound of general formula-12 in presence of base and followed by cyclizing the obtained compound in presence or absence of a coupling agent in a suitable solvent to provide the compound of formula-1,
e) optionally purifying the compound of formula-1 obtained in step-d) with a suitable solvent to provide the pure compound of formula-1.
   Wherein in step-a) the suitable oxidizing agent is selected from sodium hypochlorite in presence of a catalyst like TEMPO (2,2,6,6-tetramethyl-1-piperidinyloxy, free radical)/KBr; hydrogen peroxide, NBS-benzoyl peroxide, cumene hydroperoxide, per acids such as peracetic acid, trifluoro peracetic acid, perbenzoic acid, m-chloroperbenzoic acid (MCPBA); 4,5-dichloro-3,6-dihydroxyphthalonitrile (DDQ), 2,3,5,6-tetrachlorocyclohexa-2,5-diene-1,4-dione (Chloranil), potassium permanganate (KMnO₄), manganese dioxide and the like; in steps b) and step-d) the suitable base is selected from organic bases or inorganic bases; in step-d) the cyclization is carried out at 25-150° C.; the suitable catalyst is selected from as acetic acid, trifluoroacetic acid, p-toluene sulfonic acid, methane sulfonic acid, hydroxybenzotriazole or 2-hydroxypyridine and like; and suitable coupling agent is same as defined above;
   in steps-a) to e) the suitable solvent is selected from chloro solvents, alcohol solvents, ether solvents, ester solvents, ketone solvents, nitrile solvents, hydrocarbon solvents, polar-aprotic solvents, polar solvents such as water and/or mixtures thereof;
   in step-c) the compound of general formula-12 obtained in step-b) is optionally purified by converting it into its acid addition salts followed by treating the obtained salt with a suitable base to provide pure compound of formula-12.

Preferred embodiment of the present invention provides a novel process for the preparation of compound of formula-1 comprising of the following steps,
a) reacting the compound of formula-2a with aqueous sodium bicarbonate and sodium hypochlorite in presence of TEMPO (2,2,6,6-tetramethyl-1-piperidinyloxy, free radical)/KBr in the mixture of water and dichloromethane to provide the compound of formula-13a,
b) reacting the compound of formula-13a with (S)-2-amino butyramide in presence of Pd/C and H₂ in methanol to provide the compound of formula-12,
c) cyclizing the compound of general formula-12 in the presence acetic acid in toluene to provide the compound of formula-1,
d) purifying the compound of formula-1 obtained in step-c) with isopropyl acetate and cyclohexane to provide the pure compound of formula-1.

The ninth aspect of the present invention provides a novel process for the preparation of compound of formula-1 comprising of the following steps,
a) reacting the compound of general formula-13 with the (S)-2-amino butyramide compound of formula-18 or its salts in presence or absence of a base in a suitable solvent and a suitable reducing agent to provide the compound of general formula-22, or
   reacting the compound of general formula-13 with (S)-2-amino butyramide compound of formula-18 or its salts in the presence of a base in a suitable solvent followed by treating the obtained compound with a suitable reducing agent to provide the compound of general formula-22,
b) optionally purifying the compound of general formula-22 obtained in step-a),
c) cyclizing the compound of general formula-22 in presence of a coupling agent optionally in presence of a base in a suitable solvent to provide the compound of formula-1,
d) optionally purifying the compound of formula-1 obtained in step-c) with a suitable solvent to provide the pure compound of formula-1.
   Wherein in step-a) and step-c) the suitable base is selected from organic bases or inorganic bases;
   in step-c) the suitable coupling agent is same as defined above;
   in steps-a) to d) the suitable solvent is selected from chloro solvents, alcohol solvents, ether solvents, ester solvents, ketone solvents, nitrile solvents, hydrocarbon solvents, polar-aprotic solvents, polar solvents such as water and/or mixtures thereof.

The eighth & ninth aspects of the present invention are schematically represented as follows:

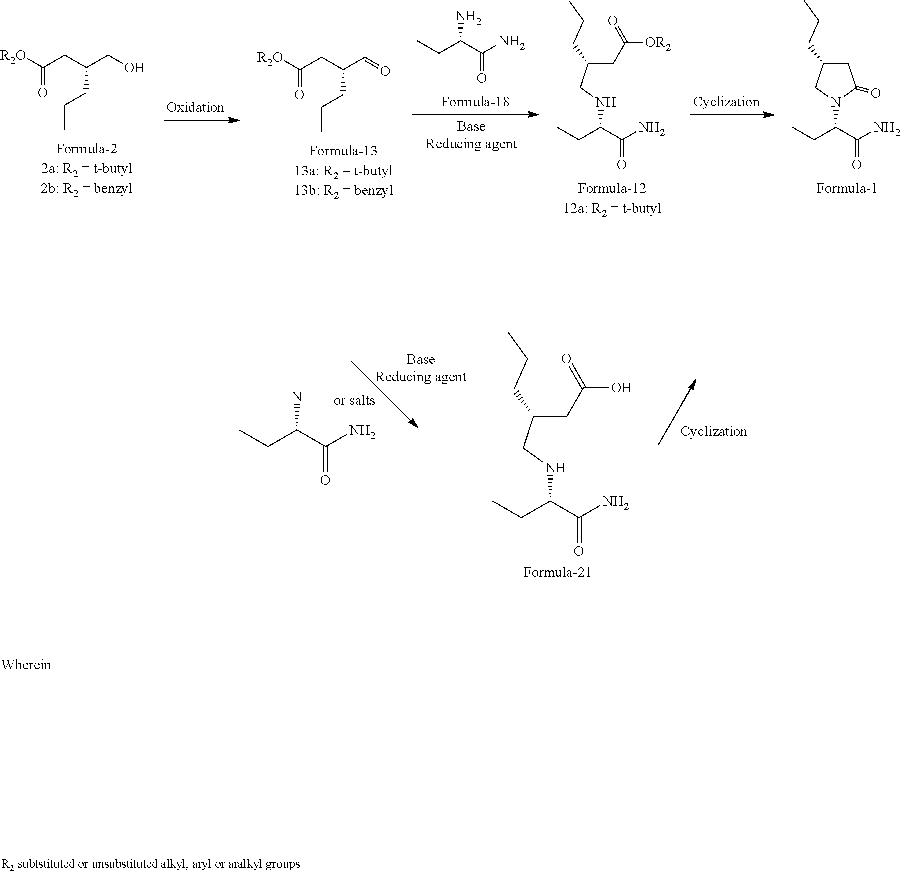

The tenth aspect the present invention provides an alternative process for the preparation of (2S)-2-[(4R)-2-oxo-4-propyltetrahydro-1H-pyrrol-1-yl] butanamide compound of formula-1 comprises the following steps:
a) reacting the compound of general formula-5 with the compound of general formula-14 in presence of a suitable base or a coupling agent in a suitable solvent to provide the compound of general formula-15,
b) reduction of the compound of general formula-15 with a suitable reducing agent in a suitable solvent to provide the compound of general formula-16,
c) cyclizing the compound of general formula-16 in presence or absence of a catalyst in a suitable solvent to provide the compound of general formula-17,
d) treating the compound of general formula-17 with a suitable amine source in a suitable solvent to provide the compound of formula-1,
e) optionally purifying the compound of formula-1 in a suitable solvent to provide pure compound of formula-1.
   Wherein in step-a) the suitable coupling agent is same as defined above; the suitable base is selected from organic bases or inorganic bases;
   in step-b) the suitable reducing agent is defined as above;
   in step-c) the cyclization is carried out at 25-150° C.; the suitable catalyst is selected from acetic acid, trifluoroacetic acid, p-toluene sulfonic acid, methane sulfonic acid, hydroxybenzotriazole or 2-hydroxypyridine and like;
   in step-d) the suitable amine source is selected from ammonia; methanolic ammonia, a mixture of formamide and metal alkoxide;
   in steps a) to e) the suitable solvent is selected from chloro solvents, alcohol solvents, ether solvents, ester solvents, ketone solvents, nitrile solvents, hydrocarbon solvents, aprotic solvents, polar solvents such as water and/or mixtures thereof.

The tenth aspect of the present invention is schematically represented as follows:

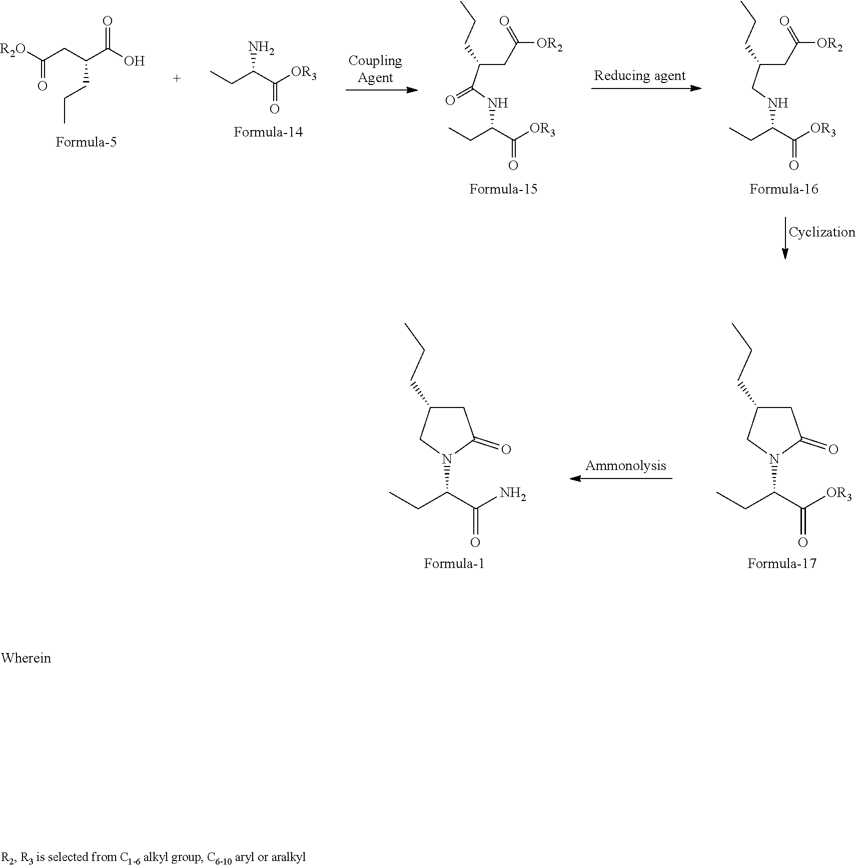

The eleventh aspect of the present invention provides a novel process for the preparation of (2S)-2-[(4R)-2-oxo-4-propyltetrahydro-1H-pyrrol-1-yl] butanamide compound of formula-1, comprising:
a) reacting the compound of general formula-5 with compound of (S)-2-amino butyramide compound of formula-18 or its salts in the presence or absence of abase or a suitable coupling agent in a suitable solvent to provide the compound of general formula-19,
b) reduction of the compound of general formula-19 in the presence of a suitable reducing agent to provide the compound of general formula-12,
c) cyclizing the compound of general formula-12 in presence or absence of catalyst in a suitable solvent to provide the compound of formula-1, or hydrolyzing the compound of general formula-12 in presence of base and followed by cyclizing the obtained compound in presence or absence of a coupling agent in a suitable solvent to provide the compound of formula-1,
d) optionally purifying the compound of formula-1 obtained in step-c) with a suitable solvent to provide the pure compound of formula-1.
   Wherein in step-a) & c) the suitable base is selected from organic or inorganic bases and coupling agent is defined as above;
   in step b) a suitable reducing agent is same as defined above;
   in step-c) the cyclization is carried out at 25-150° C.; the suitable catalyst is selected from as acetic acid, trifluoroacetic acid, p-toluene sulfonic acid, methane sulfonic acid, hydroxybenzotriazole or 2-hydroxypyridine and like;
   in steps a) to d) the suitable solvent is selected from chloro solvents, alcohol solvents, ether solvents, ester solvents, ketone solvents, nitrile solvents, hydrocarbon solvents, polar-aprotic solvents, polar solvents such as water and/or mixtures thereof.

The eleventh aspect of the present invention is schematically represented as follows:

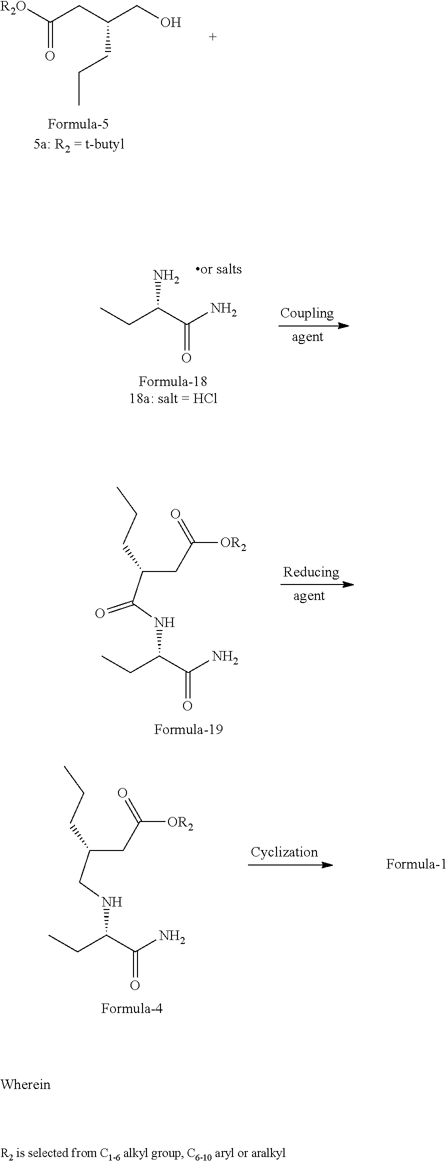

The twelfth aspect of the present invention provides a novel process for the preparation of (2S)-2-[(4R)-2-oxo-4-propyltetrahydro-1H-pyrrol-1-yl] butanamide compound of formula-1, comprising:
a) reacting the compound of general formula-13 with a suitable acid in a suitable solvent to provide the compound of formula-20,
b) reacting the compound of formula-20 with (S)-2-amino butyramide or its salts in presence of reducing agent optionally in presence of acid in a suitable solvent to provide the compound of formula-1,
c) optionally purifying the compound of formula-1 obtained in step-c) with a suitable solvent to provide the pure compound of formula-1.
   Wherein in step-a) the suitable acid is selected from organic or inorganic acids; in step b) a suitable reducing agent is same as defined above; in steps a) and b) the suitable solvent is selected from chloro solvents, alcohol solvents, ether solvents, ester solvents, ketone solvents, nitrile solvents, hydrocarbon solvents, polar-aprotic solvents, polar solvents such as water and/or mixtures thereof.
   The twelfth aspect of the present invention is schematically represented as follows:

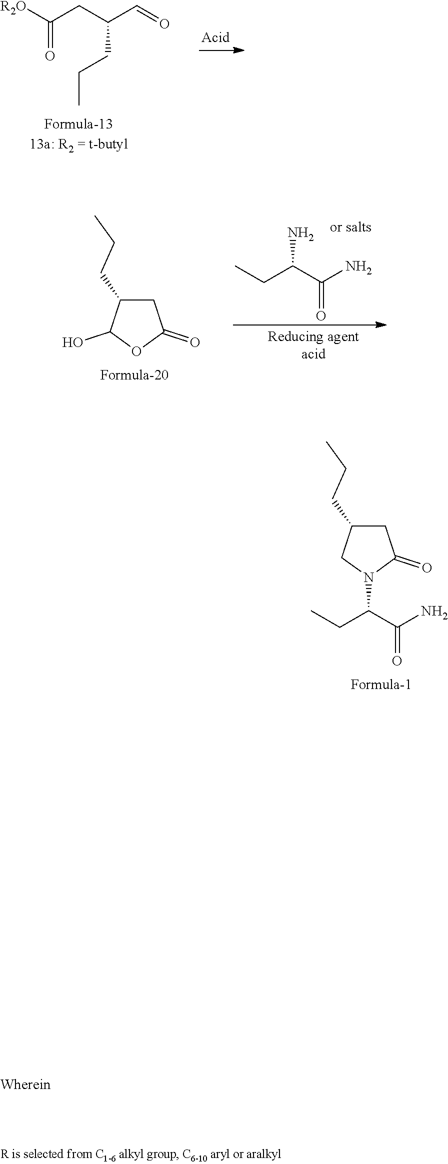

The novel intermediate compounds obtained according to the present invention are useful in the preparation of the compound of formula-1.

Another aspect of the present invention provides a process for the purification of (2S)-2-[(4R)-2-oxo-4-propyltetrahydro-1H-pyrrol-1-yl] butanamide compound of formula-1, comprising of:
a) dissolving the compound of formula-1 in a suitable solvent at a suitable temperature,
b) optionally treating the obtained solution in step-a) with carbon,
c) optionally adding a second solvent to the obtained solution in step-a) or step-b),
d) cooling the reaction mixture to a suitable temperature,
e) stirring the reaction mixture,
f) filtering the precipitated solid to provide pure compound of formula-1.
   Wherein in step-a) the suitable solvent is selected from ether solvents, alcohol solvents, ester solvents, chloro solvents or mixtures thereof, the suitable temperature is 25° C. to reflux temperature of the solvent used;
   in step-c) the second solvent is hydrocarbon solvents, preferably cyclohexane;
   in step-d) the suitable temperature is about −5° C. to 30° C.

Preferred embodiment of the present invention provides a process for the purification of (2S)-2-[(4R)-2-oxo-4-propyltetrahydro-1H-pyrrol-1-yl] butanamide compound of formula-1, comprising of:
a) dissolving the compound of formula-1 in methyl tertiary butyl ether at 50-60° C.,
b) treating the obtained solution in step-a) with carbon,
c) cooling the reaction mixture to 0-10° C.,
d) stirring the reaction mixture,
e) filtering the precipitated solid to provide pure compound of formula-1.

Another preferred embodiment of the present invention provides a process for the purification of (2S)-2-[(4R)-2-oxo-4-propyltetrahydro-1H-pyrrol-1-yl] butanamide compound of formula-1, comprising of:
a) dissolving the compound of formula-1 in isopropyl acetate at 45-65° C.,
b) treating the obtained solution in step-a) with carbon,
c) adding cyclohexane to the obtained solution in step-b),
d) cooling the reaction mixture to 5-20° C.,
e) stirring the reaction mixture,
f) filtering the precipitated solid to provide pure compound of formula-1.

(2S)-2-[(4R)-2-oxo-4-propyltetrahydro-1H-pyrrol-1-yl] butanamide compound of formula-1 obtained according to the present invention is crystalline polymorph I which is having powder X-Ray diffractogram having peaks at 8.8, 10.0, 14.9, 15.7, 17.3, 17.8, 19.0, 21.5 and 24.9±0.2 degrees of two theta and its PXRD pattern shown in figure-1 and its DSC shown in figure-2.

The crystalline polymorph I of (2S)-2-[(4R)-2-oxo-4-propyltetrahydro-1H-pyrrol-1-yl]butanamide compound of formula-1 obtained according to the present invention having purity by HPLC>99%, preferably >99.5% and having chiral purity by HPLC>99%, preferably >99.5% and more preferably >99.75%.

The structures of other isomers of the (2S)-2-[(4R)-2-oxo-4-propyltetrahydro-H-pyrrol-1-yl] butanamide compound of formula-1 are as follows:

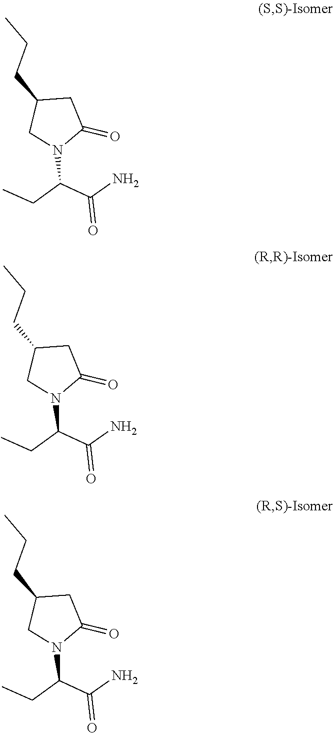

### Method of Analysis:

a) The compound of formula-1 and its related substances were analyzed by HPLC with the following chromatographic conditions:
   Apparatus: A liquid chromatograph is equipped with variable wavelength UV Detector. Column: Zodiac C8 150×4.6 mm, 3 μm; Wavelength: 210 nm; Column temperature: 25° C.; Injection volume: 10 μL; Diluent: Acetonitrile:Water (50:50 v/v); Needle wash: Diluent; Elution: Gradient; Buffer: 2.0 ml of Orthophospharic acid mix with 1000 mL of milli-Q-water and filter.
   Mobile phase-A: Buffer (100%); Mobile phase-B: Acetonitrile:Water [90:10 v/v]
b) The compound of formula-1 and its isomers were analyzed by Chiral HPLC with the following chromatographic conditions:
   Apparatus: A liquid chromatograph is equipped with variable wavelength UV Detector. Column: Chiral pack-IA-3 250×4.6 mm, 3 μm or equivalent; Wavelength: 210 nm; Column temperature: 15° C.; Injection volume: 10 μL; Diluent: n-Hexane:Ethanol (50:50 v/v); Needle wash: Diluent; Mobile phase: n-Hexane:Ethanol [80:20 v/v].
The compound of formula-1 produced by the process of the present invention is having particle size distribution of D₉₀ less than 500 μm, preferably less than 300 μm.
The compound of formula-1 produced by the present invention can be further micronized or milled to get the desired particle size. Techniques that may be used for particle size reduction include, but not limited to ball, roller and hammer mills, and jet mills. Milling or micronization may be performed before drying, or after the completion of drying of the product.
PXRD analysis of (2S)-2-[(4R)-2-oxo-4-propyltetrahydro-1H-pyrrol-1-yl]butanamide compound of formula-1 was carried out using BRUKER D8 ADVANCED/AXS X-Ray diffractometer using Cu Kα radiation of wavelength 1.5406 A° and continuous scan speed of 0.03°/min.
The process described in the present invention was demonstrated in examples illustrated below. These examples are provided as illustration only and therefore should not be construed as limitation of the scope of the invention.

### EXAMPLES

### Example-1: Preparation of (S)-3-pentanoyl-4-phenyloxazolidin-2-one [Formula-7a]

To the mixture of n-valeric acid (156.5 g) and dichloromethane (1000 ml), (S)-4-phenyloxazoldin-2-one compound of formula-6a (200 g) and 4-dimethylaminopyridine (29.9 g) were added at 25-30° C. and stirred for 10 minutes at same temperature. The reaction mixture was cooled to 5-10° C. A solution of N,N-dicyclohexylcarbodiimide (316.1 g) in 200 ml of dichloromethane was slowly added to the reaction mixture at 5-10° C. and stirred for 12 hours at the same temperature. Cooled the reaction mixture to 0-5° C. and washed the reaction mixture with aqueous HCl solution followed by water. The obtained organic layer was washed with aqueous sodium carbonate solution followed by water. Distilled off the solvent completely from the organic layer under reduced pressure and co-distilled with n-heptane. To the obtained residue 1600 ml of n-heptane was added at 25-30° C. Heated the reaction mixture to 50-55° C. and stirred for 15 minutes. The reaction mixture was slowly cooled to 0-5° C. and stirred for 2½ hours at the same temperature. Filtered the precipitated solid, washed with n-heptane and dried to get the title compound.

Yield: 243.6 g. M.R.: 42-45° C.

### Example-2: Preparation of (R)-tert-butyl 3-((S)-2-oxo-4-phenyloxazolidine-3-carbonyl) hexanoate [Formula-8a]

A solution of (S)-3-pentanoyl-4-phenyloxazolidin-2-one (60 g) in tetrahydrofuran (480 ml) was cooled to −75 to −70° C. NaHMDS (133.2 ml) was slowly added to the reaction mixture at −75 to −70° C. and stirred for 40 minutes at the same temperature. 52.1 g of t-butylbromoacetate compound of formula-10a was slowly added to the reaction mixture at −75 to −70° C. and stirred for 6 hours at same temperature. The reaction mixture was slowly quenched with aqueous ammonium chloride solution at −75 to −70° C. Raised the temperature of the reaction mixture to 25-30° C. and stirred for 20 minutes at same temperature. Separated both the organic and aqueous layers and the aqueous layer was extracted with ethyl acetate. Combined the organic layers and washed with aqueous sodium chloride solution. Distilled off the solvent completely under reduced pressure to get the title compound.

Yield: 85 g.

### Example-3: Preparation of (R)-2-(2-(tert-butoxy)-2-oxoethyl)pentanoic acid [Formula-5a]

Sodium hydroxide (13.2 g) was added to the pre-cooled mixture of water (80 ml) and tetrahydrofuran (160 ml) at 0-5° C. Hydrogen peroxide (11.3 g) was added to the reaction mixture at 0-5° C. and stirred the reaction mixture for 30 minutes at same temperature. To this reaction mixture (R)-tert-butyl-3-((S)-2-oxo-4-phenyloxazolidine-3-carbonyl)hexanoate (80 g) was added at 0-5° C. and stirred for 3 hours at same temperature. The reaction was slowly quenched with aqueous sodium thiosulfate solution at 0-5° C. Raised the temperature of the reaction mixture to 25-30° C. and water was added to the reaction mixture. The reaction mixture was washed with dichloromethane. Acidified the reaction mixture by using 5N hydrochloric acid solution. Ethyl acetate was added to the reaction mixture and separated both the organic and aqueous layers. The aqueous layer was extracted with ethyl acetate. Combined the organic layers and washed with aqueous sodium chloride solution. Distilled off the solvent completely from the organic layer to get title compound.

Yield: 28.8 g. Purity by GC: 89.33%.

### Example-4: Purification of (R)-2-(2-(tert-butoxy)-2-oxoethyl)pentanoic acid [Formula-5a] Using (S)-phenylethyl Amine

(S)-phenylethyl amine (16.2 g) was slowly added to the solution of (R)-2-(2-(tert-butoxy)-2-oxoethyl)pentanoic acid (25 g) in methyl tertiary butyl ether (250 ml) at 25-30° C. Heated the reaction mixture to 60-65° C. and stirred for 15 minutes at the same temperature. Cooled the reaction mixture to 25-30° C. and stirred for 2 hours at the same temperature. Filtered the precipitated solid and washed with methyl tertiary butyl ether. Water (125 ml) and aqueous sodium hydroxide solution were added to the above obtained compound at 25-30° C. and washed the reaction mixture with dichloromethane. Acidified the reaction mixture using 5N hydrochloric acid solution. To this reaction mixture ethyl acetate was added and separated both organic and aqueous layers. The aqueous layer was extracted with ethyl acetate. Combined the organic layers and distilled off solvent completely from the organic layer under the reduced pressure to get the pure title compound.

Yield: 16.0 g. Purity by GC: 91.69%.

### Example-5: Purification of (R)-2-(2-(tert-butoxy)-2-oxoethyl)pentanoic acid [Formula-5a] Using Benzylamine

Benzylamine (13.6 g) was slowly added to the solution of (R)-2-(2-(tert-butoxy)-2-oxoethyl)pentanoic acid (25 g) in ethyl acetate (250 ml) at 25-30° C. Heated the reaction mixture to 60-65° C. and stirred for 15 minutes at the same temperature. Cooled the reaction mixture to 5-10° C. and stirred for 3 hours at the same temperature. Filtered the precipitated solid and washed with ethyl acetate. Water (125 ml) and toluene (50 ml) were added to the above obtained compound at 25-30° C. Acidified the reaction mixture using dilute hydrochloric acid solution. Separated both organic and aqueous layers. The aqueous layer was extracted with toluene. Combined the organic layers and distilled off solvent completely under the reduced pressure to get the pure title compound.

Yield: 16.0 g. Purity by GC: 99.63%.

### Example-6: Purification of (R)-2-(2-(tert-butoxy)-2-oxoethyl)pentanoic acid [Formula-5a] Using Tertiary Butyl Amine

Tertiary butyl amine (7.2 ml) was slowly added to the solution of (R)-2-(2-(tert-butoxy)-2-oxoethyl)pentanoic acid (15 g) in ethyl acetate (150 ml) at 25-30° C. Heated the reaction mixture to 60-65° C. and stirred for 15 minutes at the same temperature. Cooled the reaction mixture to 5-10° C. and stirred for 3 hours at the same temperature. Filtered the precipitated solid and washed with ethyl acetate. Water (75 ml) and toluene (30 ml) were added to the above obtained compound at 25-30° C. Acidified the reaction mixture using dilute hydrochloric acid solution. Separated both organic and aqueous layers. The aqueous layer was extracted with toluene. Combined the organic layers and distilled off solvent completely under the reduced pressure to get the pure title compound.

Yield: 10.0 g. Purity by GC: 95.36%.

### Example-7: Preparation of (R)-tert-butyl 3-(hydroxymethyl)hexanoate [Formula-2a]

Sodium borohydride was slowly added to pre-cooled tetrahydrofuran at −5 to 0° C. and stirred for 5 minutes. BF₃-etherate (25.6 g) was slowly added to the reaction mixture at −5 to 0° C. and stirred for 15 minutes at the same temperature. (R)-2-(2-(tert-butoxy)-2-oxoethyl) pentanoic acid (23 g) was added to the reaction mixture at −5 to 0° C. and stirred for 3 hours at the same temperature. The reaction mixture was slowly quenched with water at −5 to 0° C. and raised the temperature of the reaction mixture to 25-30° C. Separated both the aqueous and organic layers and the aqueous layer was extracted with ethyl acetate. Combined the organic layers and washed with aqueous sodium chloride solution. Distilled off solvent completely from the organic layer to get title compound.

Yield: 16.2 g.

### Example-8: Preparation of (R)-ethyl 3-(bromomethyl)hexanoate [Formula-3a]

Sodium bromide (28.4 g) and trimethyl silylchloride (30 g) were added to the solution of (R)-tert-butyl 3-(hydroxymethyl)hexanoate (7 g) in ethanol (70 ml) at 25-30° C. Heated the reaction mixture to 65-70° C. and stirred the reaction mixture for 4 hours at same temperature. Distilled off the solvent from the reaction mixture under reduced pressure and cooled the obtained residue to 25-30° C. Water and ethyl acetate were added to the above residue. Separated both the organic and aqueous layers and the aqueous layer was extracted with ethyl acetate. Combined the organic layers and washed with aqueous sodium chloride solution. Distilled off the solvent completely from the organic layer under the reduced pressure to get the title compound.

Yield: 5.4 g.

### Example-9: Preparation of (2S)-2-[(4R)-2-oxo-4-propyltetrahydro-1H-pyrrol-1-yl] Butanamide [Formula-1]

Potassium iodide (3.5 g), potassium carbonate (17.5 g) and (S)-2-aminobutyramide hydrochloride (8.7 g) were added to the solution of (R)-ethyl 3-(bromomethyl)hexanoate (10 g) in the mixture of tetrahydrofuran (80 ml) and dimethyl sulfoxide (20 ml) at 25-30° C. Heated the reaction mixture to 80-85° C. and stirred for 30 hours at the same temperature. Distilled off the solvent from the reaction mixture under reduced pressure and cooled the obtained residue to 25-30° C. Water and ethyl acetate were added to the above residue. Separated both the organic and aqueous layers and the aqueous layer was extracted with ethyl acetate. Combined the organic layers and washed with aqueous sodium chloride solution. Distilled off the solvent completely from the organic layer under the reduced pressure to get the title compound. Tetrahydrofuran (80 ml) was added to the above obtained residue at 25-30° C. and heated the reaction mixture to 60-65° C. Acetic acid (0.9 ml) was slowly added to the reaction mixture at 60-65° C. and stirred for 4 hours at the same temperature. Cooled the reaction mixture to 25-30° C. Sodium carbonate followed by water was added to the reaction mixture. Filtered the reaction mixture through hyflow. Distilled off the solvent completely from the filtrate under reduced pressure. Diisopropyl ether (10 ml) was added to the obtained residue at 25-30° C. and stirred for 40 minutes at same temperature. Cooled the reaction mixture to 0-5° C. and stirred 3 hours at the same temperature. Filtered the precipitated solid, washed with diisopropyl ether and dried to get title compound.

Yield: 3.4 g. Chiral Purity: 99.93%, R,R-isomer: 0.03% & S,S-isomer: 0.03%.

### Example-10: Preparation of Benzylamine Salt of (R)-2-(2-(tert-butoxy)-2-oxoethyl)pentanoic Acid [Formula-9a]

Benzylamine (13.6 g) was slowly added to the solution of (R)-2-(2-(tert-butoxy)-2-oxoethyl)pentanoic acid (25 g) in ethyl acetate (250 ml) at 25-30° C. Heated the reaction mixture to 60-65° C. and stirred for 15 minutes at the same temperature. Cooled the reaction mixture to 5-10° C. and stirred for 3 hours at the same temperature. Filtered the precipitated solid, washed with ethyl acetate and dried to get the title compound.

Yield: 29 g. M.R.: 118-120° C. PXRD of the obtained compound is shown in figure-3.

### Example-11: Preparation of Tertiary Butyl Amine Slat of (R)-2-(2-(tert-butoxy)-2-oxoethyl)pentanoic Acid [Formula-9b]

Tertiary butyl amine (7.2 ml) was slowly added to the solution of (R)-2-(2-(tert-butoxy)-2-oxoethyl)pentanoic acid (15 g) in ethyl acetate (150 ml) at 25-30° C. Heated the reaction mixture to 60-65° C. and stirred for 15 minutes at the same temperature. Cooled the reaction mixture to 5-10° C. and stirred for 3 hours at the same temperature. Filtered the solid, washed with ethyl acetate and dried to get the title compound.

Yield: 16.3 g. M.R.: 154-156° C. PXRD of the obtained compound is shown in figure-4.

### Example-12: Preparation of (S)-Phenylethyl Amine Salt of (R)-2-(2-(tert-butoxy)-2-oxoethyl)pentanoic Acid [Formula-9c]

(S)-phenylethyl amine (16.2 g) was slowly added to the solution of (R)-2-(2-(tert-butoxy)-2-oxoethyl)pentanoic acid (25 g) in methyl tertiary butyl ether (250 ml) at 25-30° C. Heated the reaction mixture to 60-65° C. and stirred for 15 minutes at the same temperature. Cooled the reaction mixture to 25-30° C. and stirred for 2 hours at the same temperature. Filtered the precipitated solid, washed with methyl tertiary butyl ether and dried to get the title compound.

Yield: 30 g. M.R.: 118-120° C. PXRD of the obtained compound is shown in figure-5.

### Example-13: Purification of (2S)-2-[(4R)-2-oxo-4-propyltetrahydro-1H-pyrrol-1-yl] Butanamide [Formula-1]

290 g of (2S)-2-[(4R)-2-oxo-4-propyltetrahydro-1H-pyrrol-1-yl] butanamide was dissolved in 870 ml of methyl tert-butyl ether at 50-55° C. Carbon (29 g) was added to the reaction mixture at 50-55° C. and stirred for 20 minutes at the same temperature. Filtered the reaction mixture and washed with methyl tert-butyl ether. The filtrate was slowly cooled to 0-5° C. and stirred for 3 hours at the same temperature. Filtered the precipitated solid, washed with methyl tert-butyl ether and dried to get the title compound.

Yield: 220 g. Purity: 99.91%. PXRD of the obtained compound is shown in figure-1 and its DSC shown in figure-2.

### Example-14: Preparation of (R)-Tert-Butyl 3-formylhexanoate [Formula-13a]

A mixture of (R)-tert-butyl 3-(hydroxymethyl)hexanoate (10 g), dichloromethane (100 ml), potassium bromide (2.9 g) and water (20 ml) was stirred for 5 minutes at 25-30° C. Cooled the reaction mixture to 0-5° C. (2,2,6,6-tetramethylpiperidin-1-yl)oxidanyl (0.38 g) was slowly added to the reaction mixture at 0-5° C. and stirred for 15 minutes at the same temperature. Aqueous sodium bicarbonate solution and sodium hypochlorite (60 ml) were slowly added to the reaction mixture at 0-5° C. and raised the temperature to 25-30° C. Stirred the reaction mixture for 12 hours at the same temperature and reaction mixture was quenched with aqueous sodium thiosulphate solution. Separated the both organic and aqueous layers, aqueous layer was extracted with ethyl acetate and combined the organic layers. Organic layer was washed with aqueous sodium bicarbonate solution. Distilled off the solvent completely from the organic layer under reduced pressure to get the title compound.

Yield: 8.6 g

### Example-15: Preparation of (R)-Tert-Butyl 3-((((S)-1-amino-1-oxobutan-2-yl)amino) methyl)hexanoate [Formula-12a]

(R)-tert-butyl 3-formylhexanoate (2 g), methanol (20 ml), 10% palladium charcoal (0.4 g) and (S)-2-aminobutanamide (1.2 g) were charged into autoclave vessel. Hydrogen gas pressure was applied to reaction mixture at 25-30° C. Heated the reaction mixture to 45-50° C. and stirred for 4 hours at the same temperature. Cooled the reaction mixture to 25-30° C. Filtered the reaction mixture through the hy flow bed and washed with methanol. Distilled off the solvent completely from the organic layer under reduced pressure to get the title compound.

Yield: 2.1 g

### Example-16: Preparation of (2S)-2-[(4R)-2-oxo-4-propyltetrahydro-1H-pyrrol-1-yl] Butanamide [Formula-1]

A mixture of (R)-tert-butyl 3-((((S)-1-amino-1-oxobutan-2-yl)amino)methyl) hexanoate (0.5 g), toluene (10 ml) and acetic acid (2 ml) was heated to 80-85° C. and stirred for 8 hours. Acetic acid (1 ml) was added to the reaction mixture at 80-85° C. and stirred for 6 hours at the same temperature. Cooled the reaction mixture to 25-30° C. and neutralized the reaction mixture using aqueous ammonia. Separated the organic and aqueous layers and the aqueous layer was extracted with toluene. Combined the organic layers. Distilled off the solvent completely from the organic layer under reduced pressure to get title compound and co-distilled with diisopropyl ether. The obtained compound was dissolved in diisopropyl ether at 40-45° C. and stirred for 3 hours. Cooled reaction mixture to 0-5° C. Filtered the precipitated solid, washed with diisopropyl ether and dried to get the title compound.

Yield: 0.3 g.

### Example-17: Preparation of Tertiary Butyl Amine Salt of (R)-2-(2-(tert-butoxy)-2-oxo Ethyl) Pentanoic Acid [Formula-9b]

(S)-4-phenyloxazoldin-2-one (200 g) and 4-dimethylaminopyridine (30 g) were added to the solution of n-valeric acid (156.5 g) in dichloromethane (800 ml) at 25-30° C. and stirred for 10 minutes at same temperature. The reaction mixture was cooled to 5-10° C. A solution of N,N-dicyclohexylcarbodiimide (316 g) in 400 ml of dichloromethane was slowly added to the reaction mixture at 5-10° C. and stirred for 12 hours at the same temperature. Cooled the reaction mixture to 0-5° C., aqueous HCl solution was added to the reaction mixture and stirred for 20 minutes at same temperature. Filtered the reaction mixture on hy-flow bed and washed with dichloromethane. Separated the both aqueous and organic layers from the obtained filtrate. The organic layer was washed with aqueous sodium bicarbonate solution followed by water. Distilled off the solvent completely from the organic layer under reduced pressure and co-distilled with tetrahydrofuran. The obtained compound was dissolved in tetrahydrofuran (2400 ml) at 25-30° C. and cooled the reaction mixture to −75 to −70° C. NaHMDS (1240 ml) was slowly added to the reaction mixture at −75 to −70° C. and stirred for 60 minutes at the same temperature. 263 g of t-butylbromoacetate was slowly added to the reaction mixture at −75 to −70° C. and stirred for 6 hours at same temperature. The reaction mixture was slowly quenched with aqueous ammonium chloride solution at −75 to −70° C. Raised the temperature of the reaction mixture to 25-30° C. and stirred for 20 minutes at same temperature. Separated both the organic and aqueous layers and the aqueous layer was extracted with ethyl acetate. Combined the organic layers and washed with aqueous sodium bicarbonate solution followed by aqueous sodium chloride solution. Distilled off the solvent completely under reduced pressure. The above obtained compound was dissolved in tetrahydrofuran (880 ml) at 25-30° C. The obtained solution was added to the pre-cooled mixture of water (440 ml), sodium hydroxide (73.5 g) and hydrogen peroxide (125 ml) at 0-5° C. and stirred the reaction mixture for 6 hours at same temperature. The reaction mixture was slowly quenched with aqueous sodium sulphite solution at 0-5° C. Raised the temperature of the reaction mixture to 25-30° C. and water and dichloromethane were added to the reaction mixture and stirred for 10 minutes at 25-30° C. Separated the both organic and aqueous layers. Ethyl acetate was added to the aqueous layer and cooled to 0-5° C. Acidified the reaction mixture by using aqueous hydrochloric acid solution. Raised the temperature of the reaction mixture to 25-30° C. and stirred for 20 minutes at same temperature. Separated both the organic and aqueous layers. The aqueous layer was extracted with ethyl acetate. Combined the organic layers and washed with aqueous sodium chloride solution. Distilled off the solvent completely from the organic layer. The obtained compound was dissolved in ethyl acetate (800 ml) at 25-30° C. and stirred for 10 minutes same the temperature. Tertiary butyl amine (128 ml) was slowly added to the reaction mixture at 25-30° C. Heated the reaction mixture to 60-65° C. and stirred for 30 minutes at the same temperature. Cooled the reaction mixture to 25-30° C. and stirred for 3 hours at the same temperature. Filtered the precipitated solid and washed with ethyl acetate. Ethyl acetate (900 ml) was added to the obtained compound at 25-30° C. Heated the reaction mixture to 60-65° C. and stirred for 30 minutes at the same temperature. Cooled the reaction mixture to 25-30° C. and stirred for 2 hours at the same temperature. Filtered the precipitated solid, washed with ethyl acetate and dried to get the title compound.

Yield: 177 g. M.R:152-155° C. Chiral purity: 99.95%, PXRD pattern of obtained compound was depicted in figure-6 and its DSC histogram was depicted in figure-7.

### Example-18: Preparation of (2S)-2-[(4R)-2-oxo-4-propyltetrahydro-1H-pyrrol-1-yl] Butanamide [Formula-1]

To the mixture of water (500 ml) and tertiary butyl amine salt of (R)-2-(2-(tert-butoxy)-2-oxoethyl)pentanoic acid (100 g), dichloromethane (200 ml) was added at 25-30° C. Cooled the reaction mixture to 0-5° C. and acidified the reaction mixture using aqueous HCl solution at the same temperature. Separated both the organic and aqueous layers. The aqueous layer was extracted with dichloromethane. Combined the organic layers and washed with water. Distilled off the solvent completely from the organic layer and co-distilled with tetrahydrofuran. Tetrahydrofuran (360 ml) was added to the obtained compound at 25-30° C. and cooled to 0-5° C. Borane dimethylsulfide (50 g) was slowly added to the reaction mixture at 0-5° C. and stirred for 2 hours at the same temperature. Raised the temperature of the reaction mixture to 25-30° C. and stirred for 3 hours at the same temperature. The reaction mixture was slowly quenched with the aqueous potassium carbonate solution at 0-5° C. and raised the temperature of the reaction mixture to 25-30° C. and stirred for 15 minutes at the same temperature. Separated both the aqueous and organic layers and the aqueous layer was extracted with ethyl acetate. Combined the organic layers and washed with aqueous sodium chloride solution. Distilled off the solvent completely from the organic layer under reduced pressure and co-distilled with ethanol. The obtained compound was dissolved in 60 ml of ethanol at 25-30° C. Water traces removed from sodium bromide (260 g) using cyclohexane (1000 ml) by azeotropic distillation and further the solvent was completely distilled off. Ethanol (200 ml) and trimethyl silylchloride (313 g) were added to the obtained anhydrous sodium bromide at 25-30° C. and stirred for 30 minutes at the same temperature. The above obtained ethanolic solution was slowly added to this reaction mixture at 25-30° C. Heated the reaction mixture to 60-65° C. and stirred the reaction mixture for 4 hours at same temperature. Cooled the reaction mixture to 25-30° C. Water and cyclohexane were added to the above reaction mixture at 25-30° C. and stirred the reaction mixture for 20 minutes at same temperature. Separated both the organic and aqueous layers and the aqueous layer was extracted with cyclohexane. Combined the organic layers and washed with aqueous sodium bicarbonate solution followed by water. Silicagel (40 g) and carbon (3 g) were added to the obtained organic layer mixture at 25-30° C. and stirred the reaction mixture for 20 minutes at same temperature. Filtered the reaction mixture on hy-flow bed and washed with cyclohexane. Distilled off the solvent completely from the filtrate under reduced pressure and co-distilled with tetrahydrofuran. Tetrahydrofuran (600 ml) and dimethyl sulfoxide (150 ml) were added to the obtained compound at 25-30° C. Potassium iodide (24 g), potassium carbonate (121 g) and (S)-2-aminobutyramide hydrochloride (61 g) were added to the reaction mixture at 25-30° C. Heated the reaction mixture to 65-70° C. and stirred for 36 hours at the same temperature. Distilled off the solvent completely from the reaction mixture under reduced pressure. Ethyl acetate was added to the obtained compound to 25-30° C. and stirred for 10 minutes. Filtered the reaction mixture and washed with ethyl acetate. Water was added to the filtrate and stirred for 10 minutes. Separated both the organic and aqueous layers and the aqueous layer was extracted with ethyl acetate. Combined the organic layers and washed with aqueous sodium chloride solution. Acetic acid (13 g) was slowly added to the reaction mixture at 25-30° C. Heated the reaction mixture to 60-65° C. and stirred for 4 hours at the same temperature. Cooled the reaction mixture to 25-30° C. Neutralized the reaction mixture by using ammonia solution at 25-30° C. and stirred for 15 minutes. Separated both the organic and aqueous layers and the aqueous layer was extracted with ethyl acetate. Combined the organic layers. Distilled off the solvent completely organic layer and co-distilled with diisopropyl ether. Diisopropyl ether (350 ml) was added to the obtained compound at 25-30° C. Cooled the reaction mixture to 0-5° C. and stirred 5 hours at the same temperature. Filtered the precipitated solid, washed with diisopropyl ether and dried to get title compound.

Yield: 22.4 g. Chiral Purity: 99.98%.

### Example-19: Preparation of (2S)-2-[(4R)-2-oxo-4-propyltetrahydro-1H-pyrrol-1-yl] Butanamide [Formula-1]

To the mixture of water (500 ml) and tertiary butyl amine salt of (R)-2-(2-(tert-butoxy)-2-oxoethyl)pentanoic acid (100 g), dichloromethane (200 ml) was added at 25-30° C. Cooled the reaction mixture to 0-5° C. and acidified the reaction mixture using aqueous HCl solution at the same temperature. Separated both the organic and aqueous layers. The aqueous layer was extracted with dichloromethane. Combined the organic layers and washed with water. Distilled off the solvent completely from the organic layer and co-distilled with tetrahydrofuran. Tetrahydrofuran (340 ml) was added to the obtained compound at 25-30° C. and cooled to 0-5° C. Borane dimethylsulfide (50 g) was slowly added to the reaction mixture at 0-5° C. and stirred for 2 hours at the same temperature. Raised the temperature of the reaction mixture to 25-30° C. and stirred for 3 hours at the same temperature. The reaction mixture was slowly quenched with the aqueous potassium carbonate solution at 0-5° C., raised the temperature of the reaction mixture to 25-30° C. and stirred for 15 minutes at the same temperature. Separated both the aqueous and organic layers and the aqueous layer was extracted with ethyl acetate. Combined the organic layers and washed with aqueous sodium chloride solution. Distilled off the solvent completely from the organic layer under reduced pressure and co-distilled with ethanol. Ethanol (186 ml) was added to obtained compound at 25-30° C. and cooled the reaction mixture to 0-5° C. Trimethylsilyl bromide (254 g) was added to reaction mixture at 0-5° C. and stirred for 15 minutes at the same temperature. Raised the reaction mixture temperature to 50-55° C. and stirred for 5 hours at the same temperature. Cooled the reaction mixture to 25-30° C. Water and cyclohexane were added to the above reaction mixture at 25-30° C. and stirred the reaction mixture for 20 minutes at same temperature. Separated both the organic and aqueous layers and the aqueous layer was extracted with cyclohexane. Combined the organic layers and washed with aqueous sodium bicarbonate solution followed by water. Silicagel (40 g) and carbon (4 g) were added to the obtained organic layer mixture at 25-30° C. and stirred the reaction mixture for 20 minutes at same temperature. Filtered the reaction mixture on hy-flow bed and washed with cyclohexane. Distilled off the solvent completely from the filtrate under reduced pressure and co-distilled with tetrahydrofuran. Tetrahydrofuran (600 ml) and dimethyl sulfoxide (150 ml) were added to the obtained compound at 25-30° C. Potassium iodide (24 g), potassium carbonate (121 g) and (S)-2-aminobutyramide hydrochloride (61 g) were added to the reaction mixture at 25-30° C. Heated the reaction mixture to 65-70° C. and stirred for 40 hours at the same temperature. Distilled off the solvent completely from the reaction mixture under reduced pressure. Ethyl acetate was added to the obtained compound to 25-30° C. and stirred for 15 minutes. Filtered the reaction mixture and washed with ethyl acetate. Water was added to the filtrate and stirred for 10 minutes. Separated both the organic and aqueous layers and the aqueous layer was extracted with ethyl acetate. Combined the organic layers and washed with aqueous sodium chloride solution. Acetic acid (13 g) was added to the reaction mixture at 25-30° C. Heated the reaction mixture to 60-65° C. and stirred for 4 hours at the same temperature. Cooled the reaction mixture to 25-30° C., neutralized the reaction mixture by using ammonia solution at 25-30° C. and stirred for 15 minutes at the same temperature. Separated both the organic and aqueous layers and the aqueous layer was extracted with ethyl acetate. Combined the organic layers. Distilled off the solvent completely organic layer and co-distilled with diisopropyl ether. Diisopropyl ether (350 ml) was added to the obtained compound at 25-30° C. Cooled the reaction mixture to 0-5° C. and stirred 5 hours at the same temperature. Filtered the precipitated solid, washed with diisopropyl ether and dried to get title compound. Yield: 28 g; Chiral Purity: 99.98%.

### Example-20: Purification of (2S)-2-[(4R)-2-oxo-4-propyltetrahydro-1H-pyrrol-1-yl] Butanamide [Formula-1]

The mixture of (2S)-2-[(4R)-2-oxo-4-propyltetrahydro-1H-pyrrol-1-yl] butanamide (72 g) and isopropyl acetate (144 ml) was heated to 50-55° C., carbon (3.6 g) was added and stirred for 30 minutes at the same temperature. Filtered the reaction mixture on hy-flow bed and washed with mixture of cyclohexane and isopropyl acetate. Cyclohexane (720 ml) was slowly added to obtained filtrate at 10-15° C. and stirred for 3 hours at the same temperature. Filtered the precipitated solid, washed with cyclohexane and dried to get the title compound. Yield: 62 g; Purity by HPLC: 99.88% and Chiral purity: 99.99%. PXRD pattern obtained compound was similar to the figure-1.

### Example-21: Preparation of (2S)-2-[(4R)-2-oxo-4-propyltetrahydro-1H-pyrrol-1-yl] Butanamide [Formula-1]

A mixture of (R)-tert-butyl 3-((((S)-1-amino-1-oxobutan-2-yl)amino)methyl) hexanoate (2 g), toluene (10 ml), dichloromethane (10 ml) and hydroxybenzotriazole (1.42 g) was heated to 65-70° C. and stirred for 27 hours at the same temperature. Cooled the reaction mixture to 25-30° C. and extracted with ethyl acetate. Separated the both organic and aqueous layers and aqueous layer was extracted with ethyl acetate. Dried the organic layer under the reduced pressure to get the title compound.

1-39. (canceled)

40. A process for the preparation of (2S)-2-[(4R)-2-oxo-4-propyltetrahydro-H-pyrrol-1-yl] butanamide compound of formula-1, 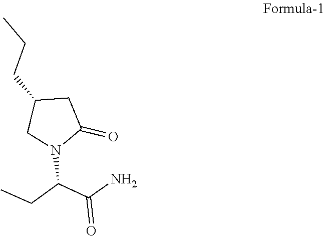
the process comprising:
a) treating the compound of formula-5 with a base in a solvent to obtain corresponding salt compound of formula-9, 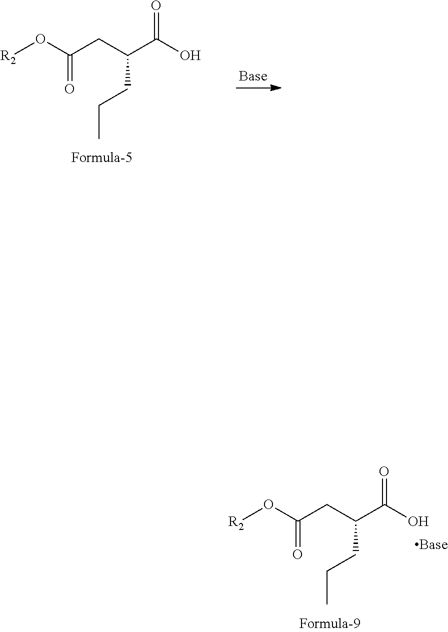
wherein R₂ is selected from the group consisting of substituted or unsubstituted alkyl, aryl, and aralkyl groups; and
b) converting the compound of formula-9 into formula-1;
wherein the suitable base is an organic amine selected from the group consisting of dimethylamine, diethylamine, diisopropyl amine, diisopropylethylamine, diisobutylamine, triethylamine, tertiary butyl amine, benzylamine, and pyridine.

41. The process of claim 40, wherein the process comprises:
a) treating the compound of formula-9 with an acid in a solvent to obtain pure compound of formula-5, 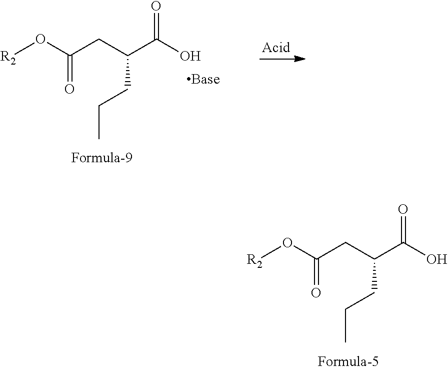
b) reduction of compound of formula-5 with a reducing agent in a solvent to provide the compound of formula-2, and 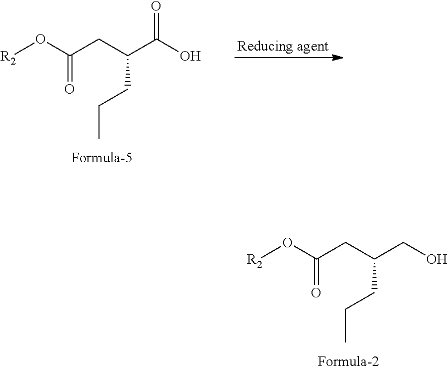
c) converting the compound of formula-2 into compound of formula-1;
wherein R₂ is selected from the group consisting of substituted or unsubstituted alkyl, aryl, and aralkyl groups.

42. The process according to claim 41, wherein the acid in step-a) is selected from inorganic or organic acid; the reducing agent in step-b) is borane dimethyl sulfide complex; and the solvent in step-a) and step-b) is selected from the group consisting of alcohol solvents, chloro solvents, ester solvents, ketone solvents, ether solvents, hydrocarbon solvents, nitrile solvents, polar solvents, water, and mixtures thereof.

43. The process according to claim 41, wherein the compound of formula-5 is formula-5a, compound of formula-9 is formula-9a or formula-9b, and compound of formula-2 is formula-2a:

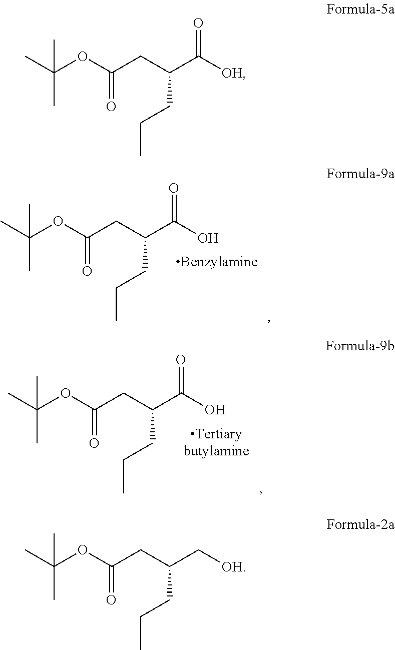

44. The process of claim 41, wherein the process further comprises:
a) reacting the compound of formula-2a with a trimethylsillyl bromide in ethanol to obtain the compound of formula-3a,
b) reacting the compound of formula-3a with (S)-2-aminobutyramide hydrochloride compound of formula-18a in presence of potassium carbonate and potassium iodide in a mixture of tetrahydrofuran and dimethyl sulfoxide to obtain the compound of formula-4a, and
c) treating the compound of formula-4a with acetic acid in ethyl acetate to obtain the compound of formula-1

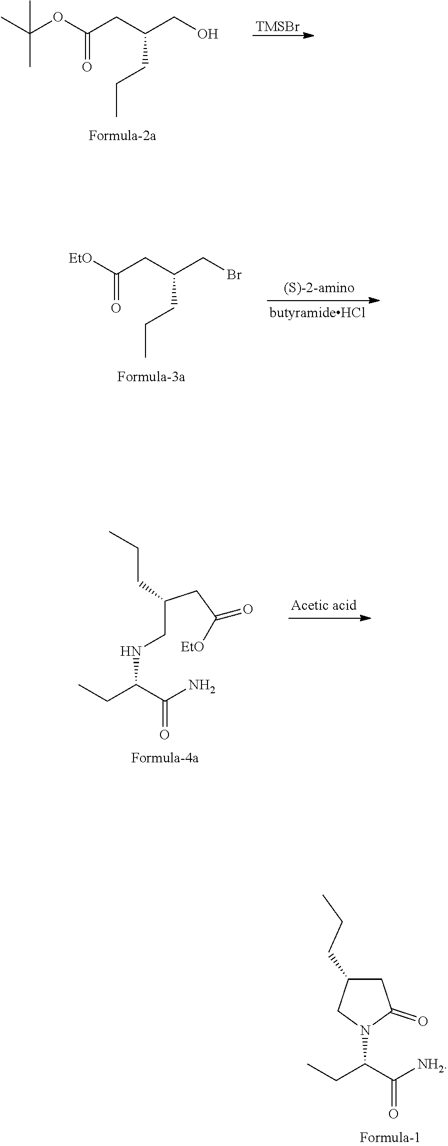 [SMILES: "TMSBr" ; CCC[C@@H](CO)CC(=O)OC(C)(C)C ; CCC[C@@H](CBr)CC(=O)OCC ; CCC[C@@H](CN[C@@H](CC)C(N)=O)CC(=O)OCC ; CCC[C@@H]1CC(=O)N([C@@H](CC)C(N)=O)C1]

45. The process of claim 40, wherein the compound of formula-5 is formula-5a, further the process for preparation of the compound of formula-5a comprises:
a) reacting the compound of formula-6a with valeric acid in presence of dicyclohexylcarbodiimide and dimethylaminopyridine in dichloromethane to obtain the compound of formula-7a,
b) reacting the compound of formula-7a with tertiary butyl bromoacetate compound of formula-10a in presence of NaHMDS in tetrahydrofuran to obtain the compound of formula-8a, and
c) treating the compound of formula-8a with hydrogen peroxide in presence of sodium hydroxide in the mixture of water and tetrahydrofuran to obtain the compound of formula-5a

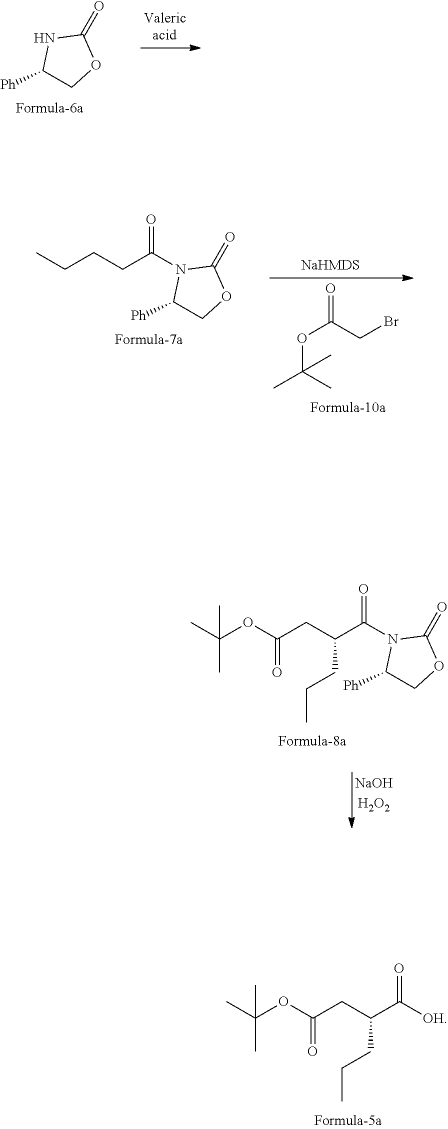

46. The process of claim 44, further comprising the purification of (2S)-2-[(4R)-2-oxo-4-propyltetrahydro-1H-pyrrol-1-yl] butanamide compound of formula-1, comprises:
a) dissolving the compound of formula-1 in a solvent to obtain a solution,
b) optionally treating the solution in step-a) with carbon,
c) optionally adding a second solvent to the solution in step-a) or step-b), and
d) filtering precipitated solid to obtain pure compound of formula-1.

47. The process according to claim 46, wherein the solvent in step-a) is selected from the group consisting of ether solvents, alcohol solvents, ester solvents, chloro solvents, and mixtures thereof, dissolving the compound at 25° C. to reflux temperature of the solvent used; the second solvent in step-c) is hydrocarbon solvents, preferably cyclohexane.

48. The process of claim 44, further comprising purification of (2S)-2-[(4R)-2-oxo-4-propyltetrahydro-1H-pyrrol-1-yl] butanamide compound of formula-1, the purification comprising:
a) dissolving the compound of formula-1 in isopropyl acetate at about 45° C. to about 65° C.,
b) adding cyclohexane to solution obtained in step-a), and
c) filtering precipitated solid to provide pure compound of formula-1.

49. The compound of formula-9 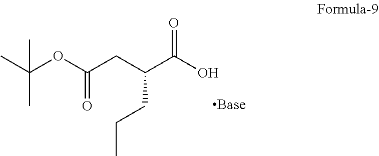
wherein the base is an organic amine selected from the group consisting of dimethylamine, diethylamine, diisopropyl amine, diisopropylethylamine, diisobutylamine, triethylamine, tertiary butyl amine, benzylamine, and pyridine.

50. (2S)-2-[(4R)-2-oxo-4-propyltetrahydro-1H-pyrrol-1-yl] butanamide of formula-1 of claim 40 having particle size distribution of D₉₀ less than 500 μm.

51. A method of treating partial-onset seizures comprising administering a therapeutically effective amount of (2S)-2-[(4R)-2-oxo-4-propyltetrahydro-1H-pyrrol-1-yl] butanamide of formula-1 of claim 40 to a subject in need thereof.
